(19) United States
(12) Patent Application Publication
(10) Pub. No.: US 2025/0195394 A1
(43) Pub. Date: Jun. 19, 2025

(54) COSMETIC EMULSION COMPRISING HYDROPHOBIC POLYMER

(71) Applicant: L'OREAL, Paris (FR)

(72) Inventors: Ritesh Kumar SINHA, Linden, NJ (US); Hy SI Bui, Piscataway, NJ (US); Xuefei Liu, Edison, NJ (US)

(73) Assignee: L'OREAL, Paris (FR)

(21) Appl. No.: 18/543,636

(22) Filed: Dec. 18, 2023

(51) Int. Cl. A61K 8/81; A61K 8/06; A61K 8/60; A61Q 1/00

(52) U.S. Cl. CPC A61K 8/8194; A61K 8/062; A61K 8/60; A61Q 1/00; A61K 2800/52; A61K 2800/54

(57) ABSTRACT

The instant disclosure is drawn to a cosmetic or personal care composition comprising: (a) hydrophobic polymer formed as a reaction product of a natural or food-derived oil and a methacrylate or acrylate polymer; (b) one or more solvents capable of solubilizing the hydrophobic polymer; (c) one or more surfactants; and (d) water. The cosmetic or personal care composition is preferably in the form of an oil-in-water emulsion. Methods for making and using the compositions are also disclosed.

### FIELD OF THE DISCLOSURE

The present disclosure relates cosmetic and personal care compositions in the form of oil-in-water emulsions containing a hydrophobic polymer formed as a reaction product of a natural or food-derived oil and a methacrylate or acrylate polymer. Also described are methods for making the compositions, and methods of using the compositions.

### BACKGROUND

Simple emulsions are dispersions of droplets of one liquid in another immiscible liquid. The droplets are typically formed by applied shear and stabilized against subsequent coalescence by a surfactant that provides an interfacial repulsion (J. Bibette, F. Leal-Calderon, and P. Poulin, REP. PROG. PHYS. 62, 969 (1999)). Two of the most common types are ‘direct’ oil-in-water (O/W) emulsions and ‘inverse’ water-in-oil (W/O) emulsions. Surfactants are amphiphilic molecules that can take many different forms, e.g., ionic (e.g. anionic, cationic, zwitterionic), nonionic (e.g. ethoxylated alkane chains), and polymeric (e.g. simple, diblock, and triblock polymers). Because they are amphiphilic, surfactants tend to preferentially adsorb onto oil-water interfaces. The relative solubility of the surfactant in the oil and the water, the concentration of the surfactant, and the degree of interfacial repulsion that the surfactant provides once it has adsorbed onto the interfaces are important factors in determining the stability and longevity of emulsions.

In general, stable emulsion systems require the use of surfactants to reduce the surface energy at an interface between a water phase and an oil phase. Many such surfactants, or emulsifiers, are known. Some types of emulsifiers, more than other types, create emulsions of greater stability. For example, O/W emulsions achieve greater stability if the emulsifier is anionic, that is a lipophilic tail attached to a hydrophilic end-group, the end-group having a net negative charge. Several lipophilic tails surround and align in the direction of an oil droplet while the hydrophilic end groups extend out into the continuous water phase, away from the oil droplet. Thus, the outer most surface of the droplet complex is negatively charged. This causes droplets to repel each other and inhibits their coalescence, which would otherwise destabilize the emulsion. Cationic emulsifiers are not generally used to stabilize an O/W emulsion. Nonionic emulsifiers may also be used to increase emulsion stability. Nonionic emulsifiers introduced into an emulsion by simple addition will migrate to the water-oil interface and lower the interfacial energy, thereby making the emulsion more stable. Low HLB nonionic emulsifiers will generally stabilize W/O emulsions, while high HLB emulsifiers will generally stabilize O/W emulsions.

The internal droplets do not share the exact same diameter, but the emulsion may be characterized as a range of droplet sizes about an average diameter. Emulsions are somewhat imprecisely classified based on the internal phase droplet size and on whether the emulsion is monodisperse or polydisperse (i.e. having one or more peak droplet diameters). Microemulsions and nano emulsions often employ an aliphatic alcohol as co-surfactant. The average oil droplet size in an O/W emulsion depends on the ratio of alcohol to other surfactant in the system. Increasing the ratio of alcohol to surfactant can decreases the average oil droplet size, which also increases the dispersion of the oil droplets and uniformity of the internal phase.

The use of emulsion is critical in the cosmetic and personal care industry. Different phases of an emulsion effectively solubilizes and transports different ingredients critical to the cosmetic product. Cosmetic compositions may be in the form of W/O or O/W emulsions, each having strengths and weakness. O/W emulsions have better mass to skin tone properties than W/O makeup emulsions. O/W emulsions generally feel lighter, cooler, and less greasy. They also tend to be easier to remove. Furthermore, O/W systems generally have better break on the skin, i.e. the composition spreads more easily and more evenly. On the other hand, W/O makeup emulsions have better or longer wear characteristics than O/W makeup emulsions, which often include a film former to improve wearability. W/O makeup emulsions also hold up to moisture better than O/W emulsions.

There remains the need to produce stable, homogeneous, cosmetic compositions with improved properties, for example, less tacky, less shiny, and less greasy during and after application. Such emulsion must be stable and retain their improved properties regardless of the addition of active ingredients.

Surprisingly, the inventors found that the hydrophobic polymer improve the stability, integrity, and cosmetic properties of emulsions formed with them. The resulting emulsions are homogeneous, stable, and impart good sensory properties, for example, they are non-tacky, non-shiny and non-greasy on the skin and hair. Therefore, they are particularly useful for cosmetic and personal care products.

### SUMMARY OF THE DISCLOSURE

The instant disclosure is drawn to stable oil-in-water emulsions particularly useful for cosmetic and personal care products. The emulsions include a unique hydrophobic polymer, one or more surfactants, one or more oils capable of solubilizing the unique hydrophobic polymer, and an aqueous phase composed primarily of water. The emulsions are surprisingly robust, versatile, and useful for incorporating and delivering cosmetic ingredients including skin and hair active agents.

Emulsions, which are dispersions of one liquid phase in another, tend to become physically unstable due to causes such as creaming, sedimentation, flocculation, phase inversion, and coalescence. The tendency toward instability is dependent, at least in part, on droplet size, droplet distribution, amount and type of emulsifiers, and mutual solubility of the two phases. Better emulsifier adsorption results in a reduction of interfacial tension, lowers the interface free energy, and thus favors emulsification and emulsion stability. The inventors found that the unique hydrophobic polymers of the emulsions of the instant disclosure positively influence (reduce) the interfacial tension between the oil droplets and the aqueous phase, contributing to smaller droplet size and improved stability.

The oil-in-water emulsions typically include:
(a) a hydrophobic polymer formed as a reaction product of a natural or food-derived oil and a methacrylate or acrylate polymer,
(b) one or more solvents capable of solubilizing the reaction product of (a);
(c) one or more surfactants; and
(d) water.

The hydrophobic polymer is a reaction product of a natural or food-derived oil and an acrylate or methacrylate polymer. According to embodiments of the disclosure, however, the hydrophobic polymer is the reaction product of a natural or food-derived oil and a methacrylate polymer. The natural or food-derived oil may be a drying oil or a semi-drying oil. Nonlimiting examples include linseed oil, sunflower oil, tung oil, fish oil, cottonseed oil, soybean oil, or combinations thereof. The methacrylate polymer can be formed from methacrylate monomers, for example, monomers selected from isobutyl methacrylate, methyl methacrylate, ethyl methacrylate, n-butyl methacrylate, and combinations thereof. In a preferred embodiment, the hydrophobic polymer is formed from a natural or food-derived oil and an isobutyl methacrylate polymer.

In various embodiments, the hydrophobic polymer is the reaction product of about 50 to about 85 parts by weight of the natural or food-derived oil and about 15 to about 50 parts by weight of the methacrylate or acrylate polymer. More specifically, the hydrophobic polymer may be the reaction product of about 72 to about 77 parts by weight of the natural or food-derived oil and about 23 to about 28 parts by weight of a methacrylate polymer. For example, the hydrophobic polymer may be the reaction product of linseed oil and poly(isobutyl methacrylate) in a suitable solvent, such as, for example, 2,2,4-trimethyl-1, 3-pentanediol monoisobutyrate. Preferably, the reaction product is formed from about 72 to about 77% of linseed oil and about 23 to about 28% of isobutyl methacrylate polymer in a suitable solvent, such as 2,2,4-trimethyl-1, 3-pentanediol monoisobutyrate.

One or more solvents capable of solubilizing the hydrophobic polymer of (a) may be a single solvent or a combination of solvents, wherein the combination of solvents is capable of solubilizing the hydrophobic polymer of (a). In various embodiments, the one or more solvents capable of solubilizing the hydrophobic polymer of (a) have a dispersion component (D), a polar component (P), and a hydrogen bonding component (H), and a distance (Ra), per Hansen Solubility Parameters of less than or equal to 13.4 MPa^{0.5}, wherein (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

Nonlimiting examples of solvents capable of solubilizing the hydrophobic polymer of (a) include polycitronellol acetate, caprylic/capric triglyceride, isododecane, isohexadecane, tetradecane, isopropyl myristate, isopropyl alcohol, octyldodecanol, ethanol, phenoxyethanol, castor oil, and mixtures thereof. In certain embodiments, polycitrnoellol acetate, caprylic/capric triglyceride, isododecane, and combinations thereof are particularly useful.

Surfactants include anionic surfactants, cationic surfactants, amphoteric (zwitterionic) surfactants, and nonionic surfactants. In various embodiments, the compositions of the instant disclosure include one or more anionic surfactants, and optionally, one or more nonionic surfactants. Furthermore, one or more of the surfactants may preferably be a biosurfactant. Nonlimiting examples of biosurfactants include glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes. Preferably, at least one of the biosurfactants is a glycolipid. Nonlimiting examples of glycolipids include sophorolipids, rhamnolipids, trehalose lipids, mannosylerythritol lipids, and combinations thereof. Rhamnolipids are particularly preferred.

Nonlimiting examples of anionic surfactants include sulfate surfactants, glutamate surfactants, acyl taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates, or combinations thereof. In various embodiments, the compositions of the instant disclosure include one or more acyl taurate surfactants.

The instant disclosure also relates to methods for making emulsions. The methods entail producing an initial composition containing the hydrophobic polymer, one or more solvents capable of dissolving the hydrophobic polymer, and one or more surfactants, wherein the initial composition includes little or no water. Typically, the amount of water in the initial composition is from about 5 to about 40 wt. %, based on the total weight of the initial composition. After forming a homogenous initial composition, the initial composition is diluted with water or an aqueous phase, resulting in an emulsion. The emulsion forms surprisingly easily without the need for high energy or high shear mixing procedures. For example, the emulsion can be formed by simply shaking or gently stirring the composition upon dilution with water.

### DETAILED DESCRIPTION OF THE DISCLOSURE

The instant disclosure is drawn to cosmetic or personal care compositions in the form of oil-in-water emulsions, to compositions used in preparing the oil-in-water emulsion, and methods of making the oil-in-water emulsions. Also described are methods for making and using cosmetic and personal care compositions. The emulsions form the cosmetic or personal care compositions and are surprisingly robust, versatile, and useful for incorporating and delivering cosmetic ingredients including skin and hair active agents. The emulsions are typically oil-in-water emulsions comprising:
(a) hydrophobic polymer formed as a reaction product of a natural or food-derived oil and a methacrylate or acrylate polymer;
(b) one or more solvents capable of solubilizing the reaction product of (a);
(c) one or more surfactants; and
(d) water.

The emulsion can be formed by preparing an initial composition having a high concentration of the hydrophobic polymer, solvents, and surfactants, with a minor amount of water. Subsequently, the initial composition is diluted with additional water.

For example, the initial composition may include:
(a) about 10 to about 40 wt. % of a reaction product of a natural or food-derived oil and a methacrylate or acrylate polymer;
(b) about 1 to about 15 wt. % one or more solvents capable of solubilizing the reaction product of (a);
(c) about 20 to about 60 wt. % of one or more surfactants; and
(d) about 15 to about 40 wt. % of water;
   wherein all percentages by weight are based on a total weight of the composition.

The initial composition is diluted with water and mixed to form the final emulsion. The dilution and mixing do not require high energy or high shear mixing or processing. For example, the emulsion forms by simply shaking the final composition or gently mixing the composition. The final composition is an oil-in-water emulsion. For instance, in preferred embodiments, the emulsion includes:
(a) about 0.5 to about 10 wt. % of a reaction product formed from a natural or food-derived oil and a methacrylate or acrylate polymer;
(b) about 0.01 to about 10 wt. % of one or more solvents capable of solubilizing the hydrophobic polymer of (a);
(c) about 1 to about 10 wt. % of one or more surfactants; and
(f) about 80 to about 90 wt. % of water;
   wherein the composition is an oil-in-water emulsion, and all percentages by weight are based on a total weight of the composition.

In a preferred embodiment, the emulsion includes a plurality of surfactants, for example, one or more biosurfactants and one or more additional surfactants such as one or more anionic surfactants. For example, the emulsion may include about 1 to about 8 wt. % of one or more biosurfactants and about 0.1 to about 6 wt. % of one or more additional surfactants, such as one or more anionic surfactants, one or more amphoteric surfactants, one or more nonionic surfactants, or combinations thereof.

For purposes of the instant disclosure, the initial composition used in to prepare final emulsions may be referred to as the “initial composition” or the “first composition.” The final emulsions are useful as cosmetic and personal care products and may be referred to as “emulsions, “cosmetic compositions” or “personal care compositions.” Throughout the disclosure, when referring to “the composition,” it is understood to mean the final emulsion (the “cosmetic composition” or the “personal care composition”) unless otherwise stated.

### (a) Hydrophobic Polymer

The hydrophobic polymer is a reaction product of a natural or food-derived oil (oil component) and an acrylate component. In particular, the natural or food-derived oil may be a drying oil, preferably linseed oil. The reaction product may include an isobutyl methacrylate backbone with a plurality of linseed oil side chains. Preferably, the reaction product is a product sold under the MYCELX® brand from MYCELX Technologies Corporation of Gainesville, Georgia. See U.S. Pat. No. 5,698,139 for a description of MYCELX materials, which is incorporated herein by reference in its entirety.

The hydrophobic polymer is comprised of an oil component and a polymer component, typically reacted in a solvent. In a preferred embodiment, the hydrophobic polymer is a reaction product of linseed oil and poly(isobutyl methacrylate), in a solvent, such as 2,2,4-trimethyl-1,3-pentanediol-monoisobutyrate.

The oil component is derived from glycerin and carboxylic acids, such as linseed fatty acid to form monoglycerides, diglycerides, and triglycerides. The oil component is preferably derived from plant/vegetable or natural origin. Vegetable oils are obtained by cold pressing the seeds of a plant to obtain the oil contained therein. Of the vegetable oils, drying oils such as linseed and tung oil, semi-drying oils such as soybean and cotton seed oil, and non-drying oils such as coconut oil may be used as the oil component. The oil component typically forms about 72% to 77%, or most preferably, 74.62%, of the hydrophobic polymer (e.g., linseed oil/isobutyl methacrylate).

The polymer component may be derived from α and β-unsaturated carbonyl compounds. The polymer component is the resultant product of a monomer which is an ester of an acrylic acid, crotonic acid, isocrotonic acid, methacrylic acid, sorbic acid, cinnamic acid, maleic acid, fumaric acid, methyl methacrylic acid, or combination thereof. Nonlimiting examples of useful polymers which cover any number of reaction possibilities between the esters of such compounds include acrylate polymers, methyl methacrylate polymers, methyl/n-butyl methacrylate polymers, methacrylate copolymers, ethyl methacrylate, n-butyl methacrylate, isobutyl methacrylate, n-butyl/isobutyl methacrylate copolymers, or combinations thereof. Preferably the polymer is poly(isobutyl methacrylate).

The hydrophobic polymer is a reaction product typically formed in a liquid solvent able to dissolve or dilute the polymer component and the resulting hydrophobic polymer. The solvent, or diluent should generally comprise any liquid or mixture of liquids that is able to dissolve or dilute the polymer and the resulting hydrophobic polymer. The solvent/diluent can control the evaporation, desired flow, and coalescing of the hydrophobic polymer. The solvent may be, for example, an aliphatic hydrocarbon, aromatic hydrocarbon, alcohols, ketones, ethers, aldehydes, phenols, carboxylic acids, carboxylates, synthetic chemicals and naturally occurring substances. Preferably the solvent is 2,2,4-trimethyl-1,3-pentanediol-monoisobutyrate. Hydrophobic polymers according to the instant disclosure and methods for making them are described, for example, in U.S. Pat. Nos. 5,437,793, 5,698,139, 5,837,146, 5,961,823, 6,180,010, 6,475,393, and 6,805,727, which are incorporated herein by reference in their entireties. The preferred hydrophobic polymer may be designated as poly(linseed oil/isobutyl methacrylate).

The amount of hydrophobic polymer in the initial composition will vary. Nonetheless, the total amount of the hydrophobic polymer in the initial composition is typically from about 10 to about 40 wt. %, based on the total weight of the initial composition. In further embodiment, the total amount of the hydrophobic polymer is from about 10 to about 35 wt. %, about 10 to about 30 wt. %, about 15 to about 40 wt. %, about 15 to about 35 wt. %, about 15 to about 30 wt. %, about 20 to about 40 wt. %, about 20 to about 35 wt. %, about 20 to about 30 wt. %, about 25 to about 40 wt. %, about 25 to about 35 wt. %, or about 25 to about 30 wt. %, based on the total weight of the initial composition.

The amount of hydrophobic polymer in the final emulsion will vary and will depend on the amount of hydrophobic polymer used in the initial composition and the amount of water used to dilute the initial composition. Nonetheless, in various embodiments, the total amount of the hydrophobic polymer in the final emulsion is from about 0.1 to about 15 wt. %, based on the total weight of the final emulsion. In further embodiments, the total amount of the hydrophobic polymer in the final emulsion is from about 0.1 to about 12 wt. %, about 0.1 to about 10 wt. %, about 0.1 to about 8 wt. %, about 0.1 to about 5 wt. %, about 0.1 to about 3 wt. %, about 0.5 to about 15 wt. %, about 0.5 to about 12 wt. %, about 0.5 to about 10 wt. %, about 0.5 to about 8 wt. %, about 0.5 to about 5 wt. %, about 0.5 to about 3 wt. %, about 1 to about 15 wt. %, about 1 to about 12 wt. %, about 1 to about 10 wt. %, about 1 to about 8 wt. %, about 1 to about 5 wt. %, about 1 to about 3 wt. %, about 2 to about 15 wt. %, about 2 to about 12 wt. %, about 2 to about 10 wt. %, about 2 to about 8 wt. %, about 2 to about 5 wt. %, based on the total weight of the final emulsion.

### (b) Solvent Capable of Solubilizing (a)

The oil phase of the emulsion includes the hydrophobic polymer of (a) dissolved in one or more solvents capable of solubilizing the hydrophobic polymer. The one or more solvent may include one or more solvents used to generate or solubilize the hydrophobic polymer, e.g., 2,2,4-trimethyl-1,3-pentanediol-monoisobutyrate. The one or more solvents may be a single solvent or a plurality of solvents. For example, in various embodiments, one or more solvents capable of solubilizing the hydrophobic polymer have a dispersion component (D), a polar component (P), a hydrogen bonding component (H), and a distance (Ra) of less than or equal to 13.4 MPa^{0.5} per the Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}.

In a preferred embodiment, the one or more solvents have a dispersion component (D), a polar component (P), a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}.

The solvent may be an oil. The term “oil” is intended to mean a non-aqueous compound, non-miscible in water, and liquid at 25° C. and atmospheric pressure (760 mmHg; 1.013×10⁵ Pa). The solvent may be a non-silicone oil (e.g., an oil that does not contain silicon atoms, and does not contain Si—O groups). Nonlimiting examples of particularly useful solvents include caprylic/capric triglyceride, isopropyl myristate, polycitronellol acetate, or combinations thereof. The solvent may include acetone. The solvent may include oleic acid. The solvents may include an oleic acid containing oil (such as a vegetable oil). Table 1, below, shows values of D, P, and H, as well as Ra for allowable ranges as well as preferred ranges, for several solvents.

TABLE 1

| | | | | Ra | Ra |
|---|---|---|---|---|---|
| | | | | (Allowable | (Preferred |
| Solvent (b) | D | P | H | Range) | Range) |
| Ethanol | 15.8 | 8.8 | 19.4 | 7.81 | 8.67 |
| Octyldodecanol | 16.1 | 2.2 | 7.4 | 6.33 | 5.17 |
| Isopropyl | 15.9 | 2.1 | 2.8 | 10.70 | 9.41 |
| Myristate | | | | | |
| Isopropyl | 15.8 | 6.1 | 6.4 | 7.02 | 5.54 |
| Alcohol | | | | | |
| Phenoxyethanol | 17.8 | 5.7 | 14.3 | 2.55 | 3.88 |
| CCTG | 18.22 | 5.39 | 14.74 | 3.38 | 4.76 |
| Castor Oil | 15.9 | 4.6 | 12 | 2.07 | 1.12 |
| Polycitronellol | 16.4 | 3 | 4.2 | 9.02 | 7.76 |
| Acetate | | | | | |
| Acetone | 15.5 | 10.4 | 7 | 8.61 | 7.38 |
| Oleic Acid | 16 | 2.8 | 6.2 | 7.27 | 5.98 |

In various embodiments, if oleic acid is utilized, at least some of the oleic acid may be provided by a vegetable oil. The vegetable oil may be a seed or nut oil. The vegetable oil may have an oleic acid content of at least 20% by weight of the vegetable oil. The vegetable oil may include sunflower oil, soybean oil, macadamia nut oil, and/or avocado oil. In some embodiments, the composition may include macadamia nut oil, and may be free, or substantially free, of other vegetable oils.

For purposes of the instant disclosure, the one or more of the solvents capable of solubilizing the hydrophobic polymer of (a) may not individually solubilize the hydrophobic polymer but when combined with other solvents, the combination solubilizes the hydrophobic polymer. Thus, when referring to a total amount of one or more solvents capable of solubilizing the hydrophobic polymer, the inclusion of all solvents that in combination solubilize the hydrophobic polymer is intended, even if one or more solvents in the combination do not individually solubilize the hydrophobic polymer.

Nonlimiting examples of solvents useful for solubilizing the hydrophobic polymer of (a), individually or in combination with other solvents, include polycitronellol acetate, caprylic/capric triglyceride, isododecane, isohexadecane, tetradecane, isopropyl myristate, octyldodecanol, ethanol, phenoxyethanol, castor oil, and mixtures thereof. In a preferred embodiment, at least one of the one or more solvents capable of solubilizing the hydrophobic polymer are selected from caprylic/capric triglyceride, polycitronellol acetate, isododecane, or mixtures thereof. In another preferred embodiment, at least one of the one or more solvents capable of solubilizing the hydrophobic polymer is polycitronellol acetate.

Nonlimiting solvents that individually or in combination with other solvents are useful for solubilizing the hydrophobic polymer of (a) include dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof.

The total amount of the one or more solvents capable of solubilizing the hydrophobic polymer of (a) in the initial composition will vary but may be from about 1 to about 99 wt. %, based on the total weight of the initial composition. For example, in various embodiments, the initial composition includes from about 1 to about 95 wt. %, about 1 to about 90 wt. %, about 1 to about 80 wt. %, about 1 to about 60 wt. %, about 1 to about 50 wt. %, about 1 to about 40 wt. %, about 1 to about 30 wt. %, about 5 to about 99 wt. %, about 5 to about 90 wt. %, about 5 to about 80 wt. %, about 5 to about 60 wt. %, about 5 to about 50 wt. %, about 5 to about 40 wt. %, about 5 to about 30 wt. %, about 10 to about 99 wt. %, about 10 to about 90 wt. %, about 10 to about 80 wt. %, about 10 to about 60 wt. %, about 10 to about 50 wt. %, about 10 to about 40 wt. %, or about 10 to about 30 wt. %, about 20 to about 99 wt. %, about 20 to about 90 wt. %, about 20 to about 80 wt. %, about 20 to about 60 wt. %, about 20 to about 50 wt. %, about 30 to about 99 wt. %, about 30 to about 90 wt. %, about 30 to about 80 wt. %, about 30 to about 60 wt. %, about 30 to about 50 wt. %, about 40 to about 99 wt. %, about 40 to about 90 wt. %, about 40 to about 80 wt. %, about 40 to about 60 wt. %, about 50 to about 99 wt. %, about 50 to about 90 wt. %, about 50 to about 80 wt. %, about 60 to about 99 wt. %, about 60 to about 90 wt. %, about 70 to about 99 wt. %, or about 70 to about 90 wt. % based on the total weight of the initial composition.

In a preferred embodiment, lower amounts of the one or more solvents capable of solubilizing the hydrophobic polymer of (a) are useful in the initial composition. For example, the initial composition may include from about 1 to about 20 wt. % of the hydrophobic polymer, based on the total weight of the initial composition. In further embodiments, the total amount of the one or more solvents in the initial composition is preferably from about 1 to about 15 wt. %, about 1 to about 12 wt. %, about 1 to about 10 wt. %, about 1 to about 8 wt. %, about 2 to about 20 wt. %, about 2 to about 15 wt. %, about 2 to about 12 wt. %, about 2 to about 10 wt. %, about 2 to about 8 wt. %, about 3 to about 20 wt. %, about 3 to about 15 wt. %, about 3 to about 12 wt. %, about 3 to about 10 wt. %, about 3 to about 8 wt. %, about 5 to about 20 wt. %, about 5 to about 15 wt. %, about 5 to about 12 wt. %, about 5 to about 10 wt. %, or about 5 to about 8 wt. %, based on the total weight of the initial composition.

The total amount of the one or more solvents capable of solubilizing the hydrophobic polymer of (a) in the final emulsion will vary, for example, based on the amount in the initial composition and based on the amount of water phase combined with the initial composition. However, the total amount of the one or more solvents in the final emulsion may be from about 1 to about 60 wt. %, based on the total weight of the final emulsion. In further embodiments, the total amount of the one or more solvents is from about 1 to about 40 wt. %, about 1 to about 20 wt. %, about 1 to about 10 wt. %, about 5 to about 60 wt. %, about 5 to about 50 wt. %, about 5 to about 40 wt. %, about 5 to about 30 wt. %, about 5 to about 20 wt. %, about 10 to about 60 wt. %, about 10 to about 50 wt. %, about 10 to about 40 wt. %, about 10 to about 30 wt. %, about 10 to about 20 wt. %, about 20 to about 60 wt. %, about 20 to about 50 wt. %, about 20 to about 40 wt. %, about 30 to about 60 wt. %, about 30 to about 50 wt. %, or about 40 to about 60 wt. %, based on the total weight of the final emulsion.

In a preferred embodiment, lower amounts of the one or more solvents capable of solubilizing the hydrophobic polymer of (a) are useful in the final emulsion. For example, the final emulsion may include from about 0.05 to about 15 wt. % of the hydrophobic polymer, based on the total weight of the final composition. In further embodiments, the final emulsion includes from about 0.05 to about 10 wt. %, about 0.05 to about 8 wt. %, about 0.05 to about 5 wt. %, about 0.05 to about 3 wt. %, about 0.1 to about 15 wt. %, about 0.1 to about 10 wt. %, about 0.1 to about 8 wt. %, about 0.1 to about 5 wt. %, about 0.1 to about 3 wt. %, about 0.5 to about 15 wt. %, about 0.5 to about 10 wt. %, about 0.5 to about 8 wt. %, about 0.5 to about 5 wt. %, or about 0.5 to about 3 wt. %, based on the total weight of the final emulsion.

### (c) Surfactant

For purposes of the instant disclosure, the term “surfactant” includes emulsifiers and detergents. Surfactants, or surface-active agents, are compounds that lower the surface tension between two liquids or between a liquid and a solid. Surfactants are amphiphilic, meaning that they contain hydrophilic (water-loving) head groups and hydrophobic (water-hating, or oil-loving) tails. Surfactants adsorb at the interface between oil and water, thereby decreasing the surface tension.

For purposes of the instant disclosure, an “emulsifier” is a surfactant that stabilizes emulsions. Emulsifiers coat droplets within an emulsion and prevent them from coming together, or coalescing. An “emulsion” is a mixture of two or more liquids, with or without an emulsifier, that are normally immiscible. One of the liquids, the “dispersed phase,” forms droplets in the other liquid, the “continuous phase.”

A “detergent” is a surfactant that has cleaning properties in dilute solutions and is typically anionic.

The surfactants can be anionic, cationic, amphoteric (zwitterionic), or nonionic. Preferably, the emulsions of the instant case include one or more surfactants selected from anionic surfactants, amphoteric (zwitterionic) surfactants, nonionic surfactants, or mixtures thereof. In various embodiments, the emulsions are preferably free or essentially free from cationic surfactants. In other embodiments, the emulsions include one or more cationic surfactants. In a preferred embodiment, the emulsions include one or more biosurfactants. In a further preferred embodiment, the emulsions contain one or more biosurfactants, one or more anionic surfactants, optionally, one or more nonionic surfactants, or mixtures thereof.

In a preferred embodiment, the compositions of the instant disclosure include a plurality of surfactants, wherein the plurality of surfactants include one or more biosurfactants and one or more surfactants other than the one or more biosurfactants. In further embodiments, the compositions of the instant disclosure include one or more biosurfactants, one or more anionic surfactants, and optionally, one or more nonionic surfactants.

The total amount of the one or more surfactants in the initial composition will vary but is typically from about 10 to about 65 wt. %, based on the total weight of the initial composition. In further embodiments, the total amount of the one or more surfactants is from about 20 to about 55 wt. %, about 20 to about 50 wt. %, about 20 to about 45 wt. %, about 25 to about 60 wt. %, about 25 to about 55 wt. %, about 25 to about 50 wt. %, about 25 to about 45 wt. %, about 30 to about 60 wt. %, about 30 to about 55 wt. %, about 30 to about 50 wt. %, about 30 to a bout 45 wt. %, about 35 to about 60 wt. %, about 35 to about 55 wt. %, about 35 to about 50 wt. %, about 35 to about 45 wt. %, about 40 to about 60 wt. %, about 40 to about 55 wt. %, about 40 to about 50 wt. %, or about 40 to about 45 wt. %, based on the total weight of the initial composition.

The total amount of the one or more surfactants in the final emulsion will vary but is typically from about 0.5 to about 20 wt. %. In further embodiments, the total amount of the one or more surfactants in the final emulsion is from about 0.5 to about 15 wt. %, about 0.5 to about 12 wt. %, about 0.5 to about 8 wt. %, about 0.5 to about 6 wt. %, about 1 to about 20 wt. %, about 1 to about 15 wt. %, about 1 to about 12 wt. %, about 1 to about 10 wt. %, about 1 to about 8 wt. %, about 1 to about 6 wt. %, about 2 to about 20 wt. %, about 2 to about 15 wt. %, about 2 to about 12 wt. %, about 2 to about 10 wt. %, about 2 to about 8 wt. %, about 2 to about 6 wt. %, about 3 to about 20 wt. %, about 3 to about 15 wt. %, about 3 to about 12 wt. %, about 3 to about 10 wt. %, about 3 to about 8 wt. %, about 3 to about 6 wt. %, about 4 to about 20 wt. %, about 4 to about 15 wt. %, about 4 to about 12 wt. %, about 4 to about 10 wt. %, about 4 to about 8 wt. %, or about 4 to about 6 wt. %, based on the total weight of the final composition.

### Biosurfactant

The compositions of the instant disclosure may optionally include one or more biosurfactants. Biosurfactants are amphiphilic molecules, for example, glycolipids (e.g., sophorolipids, rhamnolipids, cellobiose lipids, mannosylerythritol lipids and trehalose lipids), lipopeptides (e.g., surfactin, iturin, fengycin, arthrofactin and lichenysin), flavolipids, phospholipids (e.g., cardiolipins), fatty acid ester compounds, fatty acid ether compounds, and high molecular weight polymers such as lipoproteins, lipopolysaccharide-protein complexes, and polysaccharide-protein-fatty acid complexes.

Biosurfactants are environmentally friendly, biodegradable, and non-toxic and may be classified into high and low molecular weight biosurfactants. Low molecular weight biosurfactant efficiently lower surface and interfacial tension, and high molecular weight biosurfactants are more effective as emulsion-stabilizing agents. Examples of low molecular weight biosurfactants include glycolipids, such as rhamnolipids, sophorolipids, lipopeptidesm, and trehalolipids. These low molecular weight biosurfactants have hydrophilic heads comprised of sugar units linked glycosidically with hydrophobic non-polar parts. Examples of high molecular weight biosurfactants include polysaccharides, lipopolysaccharides, proteins, and lipoproteins. Polysaccharide-based biosurfactant can be classified into sorbitan esters, sucrose esters and glucose-based surfactants that include alkyl polyglycosides and fatty acid glucamides.

Nonlimiting examples of biosurfactants include liptopeptides such as surfactin; fatty acids and phospholipids, polymeric matrix biosurfactants; particulate biosurfactants; and bacterial biosurfactants composed of polysaccharides, proteins, lipopolysaccharides, lipoproteins or complex mixtures of these biopolymers.

Nonlimiting examples of commercially available biosurfactants includealkyl polyglycoside available under the trademark ECOSENSE® 3000 from Dow Chemical®; D-glucopyranose, oligomeric, decyl octyl glycosides available under the trademark GLUCOPON® 215 from BASF Corporation®; rhamnolipids available under the trademark REWOFERM® SL ONE from Evonik®; D-Glucitol, 1-deoxy-1-(methylamino)-, N-coco acyl derivatives available under the trademark GLUCOTAIN® from Clariant®; rhamnolipids from Jeneil Biotech®, and BioLoop® surfactants from Lankem® Ltd.

In one embodiment, the microbial biosurfactant is a glycolipid such as rhamnolipids (RLP), sophorolipids (SLP), trehalose lipid or mannosylerythritol lipid (MEL). The biosurfactants can be added in purified form or can be present in the microbe-based composition as a result of microbial growth. The biosurfactant may be a sophorolipid. In some embodiments, the biosurfactant can also be a lipopeptide, such as surfactin, and/or a rhamnolipid.

In some embodiments, a blend of biosurfactants is present. Preferably the blend comprises a rhamnolipid, and optionally one or both of a mannosylerythritol lipid, a surfactin or a sophorolipid. In a preferred embodiment, the microbe is a non-pathogenic strain of Pseudomonas. Preferably, the strain is a producer of rhamnolipid (RLP) biosurfactants.

Other microbial strains including, for example, other fungal strains capable of accumulating significant amounts of, for example, glycolipid-biosurfactants can be used in accordance with the subject invention. Biosurfactants useful according to the present invention include mannoprotein, beta-glucan and other metabolites that have bio-emulsifying and surface/interfacial tension-reducing properties.

In various embodiments, the one or more biosurfactants are selected from surfactin, iturin, fengycin, lichenysin, serrawettin, phospholipids, rhamnolipid, sophorolipid, trehalolipid, mannosylerythritol-lipids, cellobiolipids, lipoproteins, rubiwettins, trehalose, ornithin, pentasaccharide lipids, viscosin, bacitracin, lipopeptides, and combinations thereof. In one embodiment, the biosurfactants are selected from one or more glycolipids such as, for example, rhamnolipids, rhamnose-d-phospholipids, trehalose lipids, trehalose dimycolates, trehalose monomycolates, mannosylerythritol lipids, cellobiose lipids, ustilagic acid and/or sophorolipids.

In various embodiments, the biosurfactant has an anionic character, for example, sophorolipids, trehalolipid and rhamnolipids. Preferable are the mono-rhamnolipids and di-rhamnolipids. The preferred alkyl chain length is from C₈ to C₁₂. The alkyl chain may be saturated or unsaturated.

The term “rhamnolipids” includes compounds of the general formula (II) and salts thereof,

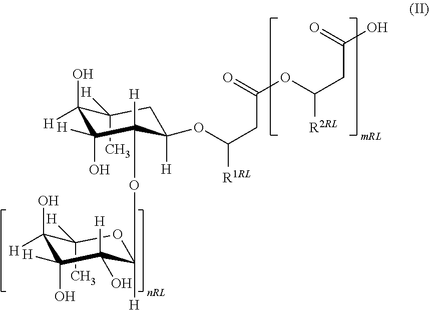

wherein,
mRL=2, 1 or 0,
nRL=1 or 0,
R^{1RL} and R^{2RL}=are independently organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CHs where o=1 to 23, preferably 4 to 12.

If nRL=1, the glycosidic bond between the two rhamnose units is preferably in the α-configuration. The optically active carbon atoms of the fatty acids are preferably present as R-enantiomers (e.g. I-3-{I-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (II) or salts thereof, where nRL=1.

The term “mono-rhamnolipid” in the context of the present invention is understood to mean compounds of the general formula (II) or salts thereof, where nRL=0.

Distinct rhamnolipids are abbreviated according to the following nomenclature: “diRL-CXCY” are understood to mean di-rhamnolipids of the general formula (II), in which one of the residues R^{1RL} and R^{2RL}=(CH₂)ₒ—CH₃ where o=X−4 and the remaining residue R¹ or R²=(CH₂)ₒ—CH₃ where o=Y−4. “monoRL-CXCY” are understood to mean mono-rhamnolipids of the general formula (II), in which one of the residues R^{1RL} and R^{2RL}=(CH.sub.₂).sub.ₒ—CH.sub.₃ where o=X−4 and the remaining residue R^{1RL} or R^{2RL}=(CH₂)ₒ—CH₃ where o=Y−4. The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “:Z”, this signifies that the respective residue R^{1RL} and/or R^{2RL} is equal to an unbranched, unsubstituted hydrocarbon residue having X−3 or Y−3 carbon atoms having Z double bonds.

Methods for preparing the relevant rhamnolipids are disclosed, for example, in EP2786743 and EP2787065, which are incorporated herein by reference in their entirety. Rhamolipids can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been Improved for higher rhamnolipid titres by genetical modification can also be used in the context of the instant invention; such cells have for example been disclosed by Lei et al. in BIOTECHNOL LETT. 2020 June; 42(6):997-1002, which is incorporated herein by reference in its entirety. The biosurfactants, in particular glycolipid surfactants, can be produced e.g. as in EP 0 499 434, U.S. Pat. No. 7,985,722, WO 03/006146, JP 60 183032, DE 19648439, DE 19600743, JP 01 304034, CN 1337439, JP 2006 274233, KR 2004033376, JP 2006 083238, JP 2006 070231, WO 03/002700, FR 2740779, DE 2939519, U.S. Pat. No. 7,556,654, FR 2855752, EP 1445302, JP 2008 062179 and JP 2007 181789, which are incorporated herein by reference in their entirety.

Rhamnolipids produced by Pseudomonas aeruginosa are commercially available from Jeneil Biotech Inc., e.g. under the tradename ZONIX®, from Logos Technologies (technology acquired by Stepan), e.g. under the tradename NATSURFACT®, from Biotensidon GmbH, e.g. under the tradename RHAPYNAL®, from AGAE® technologies, e.g. under the name R90, R95, R95Md, R95Dd, from Locus Bio-Energy Solutions and from Shanghai Yusheng Industry Co. Ltd., e.g. under the tradename BIO-201 GLYCOLIPIDS®.

The total amount of the one or more biosurfactants in the initial composition, if present, will vary but is typically from about 1 to about 60 wt. %. In further embodiments, the total amount of the one or more biosurfactants in the initial composition is from about 1 to about 50 wt. %, about 1 to about 40 wt. %, about 1 to about 30 wt. %, about 1 to about 20 wt. %, about 1 to about 10 wt. %, or about 1 to about 5 wt. %. In a further embodiment, the total amount of the one or more biosurfactants in the initial composition is from about 5 to about 60 wt. %, about 5 to about 50 wt. %, about 5 to about 40 wt. %, about 5 to about 30 wt. %, about 5 to about 20 wt. %, or about 5 to about 10 wt. %, based on the total weight of the initial composition. In yet a further embodiment, the total amount of the one or more biosurfactants in the initial compositions is from about 10 to about 60 wt. %, about 10 to about 50 wt. %, about 10 to about 40 wt. %, about 10 to about 30 wt. %, or about 10 to about 20 wt. %. In preferred embodiments, the total amount of the one or more biosurfactants in the initial composition is from about 20 to about 60 wt. %, about 20 to about 50 wt. %, about 20 to about 40 wt. %, about 30 to about 60 wt. %, about 30 to about 50 wt. %, about 30 to about 40 wt. %, about 25 to about 50 wt. %, about 25 to about 45 wt. %, about 25 to about 40 wt. %, or about 25 to about 35 wt. %, based on the total weight of the initial composition.

The total amount of the one or more biosurfactants in the final emulsion, if present, will vary but is typically from about 0.1 to about 20 wt. %. In further embodiments, the total amount of the one or more biosurfactants in the final emulsion is from about 0.1 to about 15 wt. %, about 0.1 to about 10 wt. %, or about 0.1 to about 5 wt. %. In a further embodiment, the total amount of the one or more biosurfactants in the final emulsion is from about 0.5 to about 20 wt. %, about 0.5 to about 15 wt. %, about 0.5 to about 10 wt. %, or about 0.5 to about 5 wt. %, based on the total weight of the final emulsion. In yet a further embodiment, the total amount of the one or more biosurfactants in the final emulsion is from about 1 to about 20 wt. %, about 1 to about 15 wt. %, about 1 to about 10 wt. %, or about 1 to about 5 wt. %, based on the total weight of the final emulsion. In preferred embodiments, the total amount of the one or more biosurfactants in the final emulsion is from about 2 to about 20 wt. %, about 2 to about 15 wt. %, about 2 to about 10 wt. %, about 2 to about 5 wt. %, about 3 to about 20 wt. %, about 3 to about 15 wt. %, about 3 to about 10 wt. %, about 3 to about 5 wt. %, about 2 to about 8 wt. %, about 2 to about 6 wt. %, about 3 to about 8 wt. %, or about 3 to about 6 wt. %, based on the total weight of the final emulsion.

### Anionic Surfactants

In various embodiment, the compositions of the instant disclosure include one or more anionic surfactants. Common anionic surfactants include sulfate surfactants, for example, sodium lauryl sulfate and sodium laureth ether sulfate, which may be used. In various embodiments, the one or more anionic surfactants, if present, are non-sulfate anionic surfactants. Useful non-sulfate anionic surfactants include, but are not limited to, alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, and a mixture thereof. In some cases, however, acyl taurates are preferred and therefore the one or more non-sulfate anionic surfactants include at least one acyl taurate. In other cases, acyl isethionates are preferred and therefore the one or more non-sulfate anionic surfactants include at least one acyl isethionate.

In yet other cases, a combination of acyl taurates and acyl isethionates may be used. Thus, the cleansing compositions may include two or more non-sulfate anionic surfactants comprising anionic surfactants selected from acyl taurates, acyl isethionates, or combinations thereof.

The total amount of the one or more anionic surfactants in the compositions of the instant disclosure, if present, will vary.

The total amount of the one or more anionic surfactants added to the initial composition may be from about 0.01 to about 15 wt. %, based on the total weight of the initial composition. In further embodiments, the initial composition includes from about 0.01 to about 10 wt. %, about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more anionic surfactants, based on the total weight of the initial composition.

The total amount of the one or more anionic surfactants in the final emulsions may be from about 0.01 to about 10 wt. %, based on the total weight of the final emulsion. In further embodiments, the emulsions includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more anionic surfactants, based on the total weight of the emulsion.

Non-limiting examples of non-sulfate anionic surfactants are provided below.

### (a) Acyl Isethionates

Non-limiting examples of useful acyl isethionates include those of formula (III) and (IV):

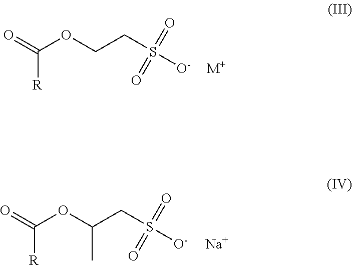

wherein R, R¹, R² and R³ are each independently selected from H or an alkyl chain having 1-24 carbon atoms, said chain being saturated or unsaturated, linear or branched, and X is COO⁻ or SO₃⁻. Although sodium is shown as the cation in formulae (III) and (IV), the cation for both formula (III) and formula (IV) may be an alkali metal ion such as sodium or potassium, ammonium ions, or alkanolammonium ions such as monoethanolammonium or triethanolammonium ions. Non-limiting examples of acyl isethionates include sodium isethionate, sodium cocoyl isethionate, sodium lauroyl methyl isethionate, and sodium cocoyl methyl isethionate. In some embodiments, a combination of sodium isethionate and sodium cocoyl isethionate are preferable.

### (b) Alkyl Sulfonates

Examples of alkyl sulfonates include alkyl aryl sulfonates, primary alkane disulfonates, alkene sulfonates, hydroxyalkane sulfonates, alkyl glyceryl ether sulfonates, alpha-olefinsulfonates, sulfonates of alkylphenolpolyglycol ethers, alkylbenzenesulfonates, phenvlalkanesulfonates, alpha-olefinsulfonates, olefin sulfonates, alkene sulfonates, hydroxyalkanesulfonates and disulfonates, secondary alkanesulfonates, paraffin sulfonates, ester sulfonates, sulfonated fatty acid glycerol esters, and alpha-sulfo fatty acid methyl esters including methyl ester sulfonate.

In some instances, an alkyl sulfonate of formula (V) is particularly useful.

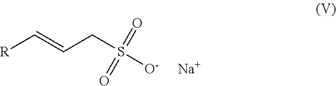

R is selected from H or alkyl chain that has 1-24 carbon atoms, preferably 6-24 carbon atoms, more preferably, 8 to 20 carbon atoms, said chain being saturated or unsaturated, linear or branched. Sodium is shown as the cation in the above formula (V) but the cation may be an alkali metal ion such as sodium or potassium, ammonium ions, or alkanolammonium ions such as monoethanolammonium or triethanolammonium ions. In some instances, the alkyl sulfonate(s) are selected from C₈-C₁₆ alkyl benzene sulfonates, C₁₀-C₂₀ paraffin sulfonates, C₁₀-C₂₄ olefin sulfonates, salts thereof, and mixtures thereof. C₁₀-C₂₄ olefin sulfonates may be particularly preferred. A non-limiting example of a C₁₀-C₂₄ olefin sulfonate that can be used in the instant compositions is sodium C₁₄-C₁₆ olefin sulfonate.

### (c) Alkyl Sulfosuccinates

Non-limiting examples of useful sulfosuccinates include those of formula (VI):

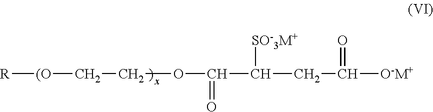

wherein R is a straight or branched chain alkyl or alkenyl group having 10 to 22 carbon atoms, preferably 10 to 20 carbon atoms, X is a number that represents the average degree of ethoxylation and can range from 0 to about 5, preferably from 0 to about 4, and most preferably from about 2 to about 3.5, and M and M′ are monovalent cations which can be the same or different from each other. Preferred cations are alkali metal ions such as sodium or potassium, ammonium ions, or alkanolammonium ions such as monoethanolammonium or triethanolammonium ions.

Non-limiting examples of alkyl sulfosuccinates salts include disodium oleamido MIPA sulfosuccinate, disodium oleamido MEA sulfosuccinate, disodium lauryl sulfosuccinate, disodium laureth sulfosuccinate, diammonium lauryl sulfosuccinate, diammonium laureth sulfosuccinate, dioctyl sodium sulfosuccinate, disodium oleamide MEA sulfosuccinate, sodium dialkyl sulfosuccinate, and a mixture thereof. In some instances, disodium laureth sulfosuccinate is particularly preferred.

### (d) Alkyl Sulfoacetates

Non-limiting examples of alkyl sulfacetates includes, for example, alkyl sulfoacetates such as C₄-C₁₈ fatty alcohol sulfoacetates and/or salts thereof. A particularly preferred sulfoacetate salt is sodium lauryl sulfoacetate. Useful cations for the salts include alkali metal ions such as sodium or potassium, ammonium ions, or alkanolammonium ions such as monoethanolammonium or triethanolammonium ions.

### (e) Alkoxylated Monoacids

Non-limiting examples of alkoxylated monoacids include compounds corresponding to formula (VII):

RO[CH2O]u[(CH2)xCH(R′)(CH2)y(CH2)zO]v[CH2CH2O]wCH2COOH (VII)
wherein:
R is a hydrocarbon radical containing from about 6 to about 40 carbon atoms;
u, v and w, independently of one another, represent numbers of from 0 to 60;
x, y and z, independently of one another, represent numbers of from 0 to 13;
R′ represents hydrogen, alkyl, and
the sum of x+y+z>0; Compounds corresponding to formula (VII) can be obtained by alkoxylation of alcohols ROH with ethylene oxide as the sole alkoxide or with several alkoxides and subsequent oxidation. The numbers u, v, and w each represent the degree of alkoxylation. Whereas, on a molecular level, the numbers u, v and w and the total degree of alkoxylation can only be integers, including zero, on a macroscopic level they are mean values in the form of broken numbers.

In formula (VII), R is linear or branched, acyclic or cyclic, saturated or unsaturated, aliphatic or aromatic, substituted or unsubstituted. Typically, R is a linear or branched, acyclic C₆-C₄₀ alkyl or alkenyl group or a C₁-C₄₀ alkyl phenyl group, more typically a C₈-C₂₂ alkyl or alkenyl group or a C₄-C₁₈ alkyl phenyl group, and even more typically a C₁₂-C₁₈ alkyl group or alkenyl group or a C₆-C₁₆ alkyl phenyl group; u, v, w, independently of one another, is typically a number from 2 to 20, more typically a number from 3 to 17 and most typically a number from 5 to 15; x, y, z, independently of one another, is typically a number from 2 to 13, more typically a number from 1 to 10 and most typically a number from 0 to 8.

Suitable alkoxylated monoacids include, but are not limited to: Butoxynol-5 Carboxylic Acid, Butoxynol-19 Carboxylic Acid, Capryleth-4 Carboxylic Acid, Capryleth-6 Carboxylic Acid, Capryleth-9 Carboxylic Acid, Ceteareth-25 Carboxylic Acid, Coceth-7 Carboxylic Acid, C₉-C₁₁ Pareth-6 Carboxylic Acid, C₁₁-C₁₅ Pareth-7 Carboxylic Acid, C₁₂-C₁₃ Pareth-5 Carboxylic Acid, C₁₂-C₁₃ Pareth-8 Carboxylic Acid, C₁₂-C₁₃ Pareth-12 Carboxylic Acid, C₁₂-C₁₅ Pareth-7 Carboxylic Acid, C₁₂-C₁₅ Pareth-8 Carboxylic Acid, C₁₄-C₁₅ Pareth-8 Carboxylic Acid, Deceth-7 Carboxylic Acid, Laureth-3 Carboxylic Acid, Laureth-4 Carboxylic Acid, Laureth-5 Carboxylic Acid, Laureth-6 Carboxylic Acid, Laureth-8 Carboxylic Acid, Laureth-10 Carboxylic Acid, Laureth-11 Carboxylic Acid, Laureth-12 Carboxylic Acid, Laureth-13 Carboxylic Acid, Laureth-14 Carboxylic Acid, Laureth-17 Carboxylic Acid, PPG-6-Laureth-6 Carboxylic Acid, PPG-8-Steareth-7 Carboxylic Acid, Myreth-3 Carboxylic Acid, Myreth-5 Carboxylic Acid, Nonoxynol-5 Carboxylic Acid, Nonoxynol-8 Carboxylic Acid, Nonoxynol-10 Carboxylic Acid, Octeth-3 Carboxylic Acid, Octoxynol-20 Carboxylic Acid, Oleth-3 Carboxylic Acid, Oleth-6 Carboxylic Acid, Oleth-10 Carboxylic Acid, PPG-3-Deceth-2 Carboxylic Acid, Capryleth-2 Carboxylic Acid, Ceteth-13 Carboxylic Acid, Deceth-2 Carboxylic Acid, Hexeth-4 Carboxylic Acid, Isosteareth-6 Carboxylic Acid, Isosteareth-11 Carboxylic Acid, Trudeceth-3 Carboxylic Acid, Trideceth-6 Carboxylic Acid, Trideceth-8 Carboxylic Acid, Trideceth-12 Carboxylic Acid, Trideceth-3 Carboxylic Acid, Trideceth-4 Carboxylic Acid, Trideceth-7 Carboxylic Acid, Trideceth-15 Carboxylic Acid, Trideceth-19 Carboxylic Acid, Undeceth-5 Carboxylic Acid and mixtures thereof. In some cases, preferred ethoxylated acids include Oleth-10 Carboxylic Acid, Laureth-5 Carboxylic Acid, Laureth-11 Carboxylic Acid, and a mixture thereof.

### (f) Acyl Amino Acids

Acyl amino acids that may be used include, but are not limited to, amino acid surfactants based on alanine, arginine, aspartic acid, glutamic acid, glycine, isoleucine, leucine, lysine, phenylalanine, serine, tyrosine, valine, sarcosine, threonine, and taurine. The most common cation associated with the acyl amino acid can be sodium or potassium. Alternatively, the cation can be an organic salt such as triethanolamine (TEA) or a metal salt. Non-limiting examples of acyl amino acids include those of formula (VIII):

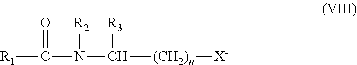

wherein R, R¹, R² and R³ are each independently selected from H or an alkyl chain having 1-24 carbon atoms, said chain being saturated or unsaturated, linear or branched, and X is COO⁻ or SO₃⁻.

### (g) Acyl Taurates

Non-limiting examples of acyl taurates include those of formula (IX):

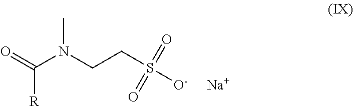

wherein R, R¹, R² and R³ are each independently selected from H or an alkyl chain having 1-24 carbon atoms, or from 6-20 carbon atoms, or from 8 to 16 carbon atoms, said chain being saturated or unsaturated, linear or branched, and X is COO⁻ or SO₃⁻. Non-limiting examples of acyl taurate salts include sodium cocoyl taurate, sodium methyl cocoyl taurate, sodium lauroyl taurate, and sodium methyl lauroyl taurate.

### (h) Acyl Glycinates

Non-limiting examples of acyl glycinates include those of formula (X):

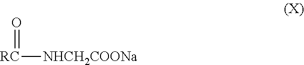

wherein R is an alkyl chain of 8 to 16 carbon atoms. Although sodium is shown as the cation in the above formula (X), the cation may be an alkali metal ion such as sodium or potassium, ammonium ions, or alkanolammonium ions such as monoethanolammonium or triethanolammonium ions. Non-limiting examples of acyl glycinates include sodium cocoyl glycinate, sodium lauroyl glycinate, sodium myristoyl glycinate, potassium lauroyl glycinate, and potassium cocoyl glycinate, and in particular sodium cocoyl glycinate.

### (i) Acyl Glutamates

Non-limiting examples of acyl glutamates include those of formula (XI):

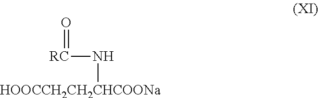

wherein R is an alkyl chain of 8 to 16 carbon atoms. Sodium is shown as the cation in the above formula (XI) but the cation may be an alkali metal ion such as sodium or potassium, ammonium ions, or alkanolammonium ions such as monoethanolammonium or triethanolammonium ions. Non-limiting examples of acyl gluatamtes include dipotassium capryloyl glutamate, dipotassium undecylenoyl glutamate, disodium capryloyl glutamate, disodium cocoyl glutamate, disodium lauroyl glutamate, disodium stearoyl glutamate, disodium undecylenoyl glutamate, potassium capryloyl glutamate, potassium cocoyl glutamate, potassium lauroyl glutamate, potassium myristoyl glutamate, potassium stearoyl glutamate, potassium undecylenoyl glutamate, sodium capryloyl glutamate, sodium cocoyl glutamate, sodium lauroyl glutamate, sodium myristoyl glutamate, sodium olivoyl glutamate, sodium palmitoyl glutamate, sodium stearoyl glutamate, sodium undecylenoyl glutamate, triethanolamine mono-cocoyl glutamate, triethanolamine lauroylglutamate, and disodium cocoyl glutamate. In some cases, sodium stearoyl glutamate is particularly preferred.

### (j) Acyl Sarcosinates:

Non-limiting examples of acyl sarcosinates include potassium lauroyl sarcosinate, potassium cocoyl sarcosinate, sodium cocoyl sarcosinate, sodium lauroyl sarcosinate, sodium myristoyl sarcosinate, sodium oleoyl sarcosinate, sodium palmitoyl sarcosinate, and ammonium lauroyl sarcosinate.

### Amphoteric Surfactants

The compositions of the instant disclosure may optionally include one or more amphoteric surfactants. Nonlimiting examples of amphoteric surfactants include betaines, alkyl amphoacetates and alkyl amphodiacetates, alkyl sulltaines, alkyl amphopropionates, and combinations thereof.

The total amount of the one or more amphoteric surfactants added to the initial composition may be from about 0.01 to about 15 wt. %, based on the total weight of the initial composition. In further embodiments, the initial composition includes from about 0.01 to about 15 wt. %, about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more amphoteric surfactants, based on the total weight of the initial composition.

The total amount of the one or more amphoteric surfactants in the final emulsions may be from about 0.01 to about 10 wt. %, based on the total weight of the final emulsion. In further embodiments, the emulsions includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more amphoteric surfactants, based on the total weight of the emulsion.

### (a) Betaines

The one or more betaine surfactants may be in the form of a salt in the cleansing composition or before addition to the cleansing composition. The betaine surfactants may be derived from a variety of natural oils or fatty acids.

In some embodiments, exemplary useful betaines include, but are not limited to, those of the following formulae (la-Id):

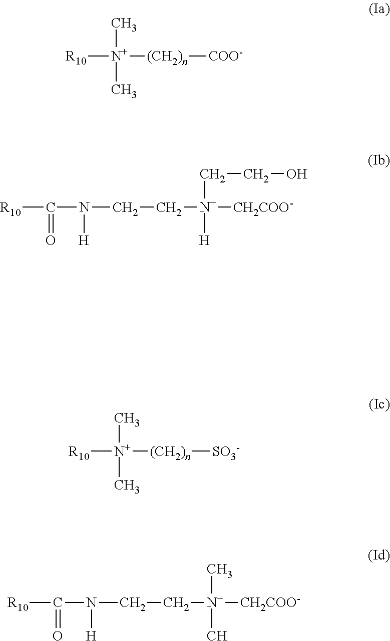

wherein:
R₁₀ is an alkyl group having from 8-18 carbon atoms; and
n is an integer from 1 to 3.

Particularly useful betaines include, for example, coco-betaine, cocamidopropyl betaine, lauryl betaine, laurylhydroxy sulfobetaine, lauryldimethyl betaine, cocamidopropyl hydroxysultaine, behenyl betaine, capryl/capramidopropyl betaine, lauryl hydroxysultaine, stearyl betaine, or mixtures thereof. Typically, at least one betaine compound is selected from coco betaine, cocamidopropyl betaine, behenyl betaine, capryl/capramidopropyl betaine, and lauryl betaine, and mixtures thereof. In one embodiment, preferred betaines include coco-betaine and cocamidopropyl betaine.

### (b) Alkyl Amphoacetates and Alkyl Amphodiacetates

By way of example only, useful alkyl amphoacetates and alkyl amphodiacetates include those of Formula (IIa) or (IIb):

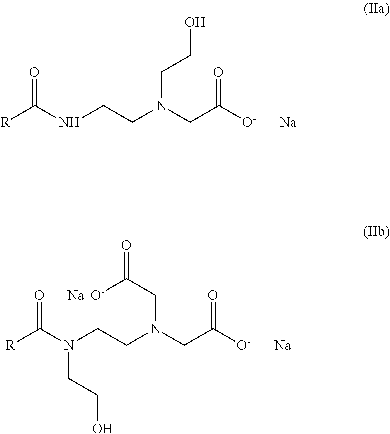

wherein R is an alkyl group having 8-18 carbon atoms.

Although sodium is shown as the cation in the above formulae, the cation may be any alkali metal ion, such as sodium or potassium, an ammonium ion, or an alkanolammonium ion such as monoethanolammonium or triethanolammonium ions. A non-limiting example is sodium lauroamphoacetate.

Additional non-limiting examples of alkyl amphoacetates and alkyl amphodiacetates include those of formula (IIc):

Ra′—CON(Z)CH2—(CH2)m′—N(B)(B′) (IIc)
wherein:
   B represents —CH2CH2OX′, with X′ representing —CH2-COOH, CH2—COOZ′, —CH2CH2—COOH, —CH2CH2—COOZ′, or a hydrogen atom;
   B′ represents —CH2)z—Y′, with z=1 or 2, and Y′ representing —COOH, —COOZ′, —CH2—CHOH—SO3H or —CH2—CHOH—SO3Z′;
   m′ is equal to 0, 1 or 2;
   Z represents a hydrogen atom or a hydroxyethyl or carboxymethyl group;
   Z′ represents an ion resulting from an alkali or alkaline-earth metal, such as sodium, potassium or magnesium; an ammonium ion; or an ion resulting from an organic amine and in particular from an amino alcohol, such as monoethanolamine, diethanolamine and triethanolamine, monoisopropanol-amine, diisopropanolamine or triisopropanolamine, 2-amino-2-methyl-1-propanol, 2-amino-2-methyl-1,3-propanediol and tris(hydroxy-methyl)aminomethane; and
Ra′ represents a (C10-C30)alkyl or alkenyl group of an acid Ra′COOH preferably present in hydrolyzed linseed oil or coconut oil, an alkyl group, in particular a C17 alkyl group, and its iso form, or an unsaturated C17 group.

Exemplary compounds of formula (Ic) include (C8-C20)alkylamphoacetates and (C8-C20)alkylamphodiacetates, such as disodium cocoamphodiacetate, disodium lauroamphodiacetate, disodium caprylamphodiacetate, disodium caprylamphodiacetate, disodium cocoamphodipropionate, disodium lauroamphodipropionate, disodium caprylampho-dipropionate, disodium caprylomphodipropionate, lauroamphodipropionic acid, or cocoamphodipropionic acid. For example, disodium cocoamphodiacetate supplied by Rhodia under the name MIRANOLI C₂M can be used.

### (c) Alkyl Sulltaines

Non-limiting examples of alkyl sultaines include hydroxyl sultaines of the following formula (IId)

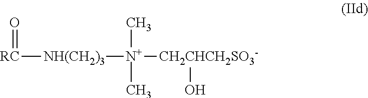

wherein R is an alkyl group having 8-18 carbon atoms. More specific examples include, but are not limited to cocamidopropyl hydroxysultaine, lauryl hydroxysultaine, and a mixture thereof.

### (d) Alkyl Amphopropionates

Non-limiting examples of alkyl amphopropionates include cocoamphopropionate, cornamphopropionatecaprylamphopropionate, cornamphopropionate,caproamphopropionate, oleoamphopropionate, isostearoamphopropionate, stearoamphopropionate, lauroamphopropionate, salts thereof, and a mixture thereof.

### Nonionic Surfactants

In various embodiments, the compositions of the instant disclosure include one or more nonionic surfactants. Nonlimiting examples of useful nonionic surfactants include alkoxylated fatty alcohols, alkoxylated polyol esters, alkoxylated glycerides, glucosides, alkanolamides, sorbitan derivatives, or combinations thereof.

The total amount of the one or more nonionic surfactants added to the initial composition may be from about 0.01 to about 15 wt. %, based on the total weight of the initial composition. In further embodiments, the initial composition includes from about 0.01 to about 10 wt. %, about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 15 wt. %, about 1 to about 10 wt. %, about 1 to about 8 wt. %, about 1 to about 6 wt. %, about 1 to about 5 wt. %, or about 1 to about 3 wt. % of the one or more nonionic surfactants, based on the total weight of the initial composition.

The total amount of the one or more nonionic surfactants in the final emulsions may be from about 0.01 to about 10 wt. %, based on the total weight of the final emulsion. In further embodiments, the emulsions includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more nonionic surfactants, based on the total weight of the emulsion.

Nonionic surfactants may optionally be alkoxylated. The alkoxylated nonionic surfactants may be chosen from alkoxylated alcohols, alkoxylated fatty alcohols, alkoxylated polyol esters such as polyethylene glycol ethers of fatty alcohols, polyethylene glycol ethers of esters, and polyethylene glycol ethers of glycerides, and mixtures thereof. Non-limiting examples of polyethylene glycol ethers of esters include ethoxylated fatty esters. Further discussion of non-limiting examples of the alkoxylated nonionic surfactants are provided below. In some instances, the alkoxylated nonionic surfactants are chosen from PEG-55 propylene glycol oleate, PEG-6 propylene glycol caprylate/caprate, PEG-8 propylene glycol cocoate, PEG-55 propylene glycol oleate, PEG-75 propylene glycol stearate, PEG-25 propylene glycol stearate, PEG-7 glyceryl cocoate, PEG-30 glyceryl cocoate, laureth-2, laureth-3, laureth-4, PEG-200 glyceryl stearate, PEG-120 propylene glycol stearate, PEG-6 Caprylic/Capric Glycerides, and a mixture thereof.

“Alkoxylated nonionic surfactant” as used herein means a compound having at least one alkoxylated portion (—(CH2)nO—, where n is an integer from 1 to 300, preferably 2 to 200, or more preferably 2 to 150, even more preferably 2 to 120, or most preferably, 2 to 100).

### (a) Alkoxylated Fatty Alcohols

“Alkoxylated fatty alcohol” as used herein means a compound having at least one fatty portion (8 carbon atoms or more) and at least one alkoxylated portion (—(CH₂)nO—, where n is an integer of 1 or more). The alkoxylated fatty alcohols of the present invention preferably have an HLB (hydrophilic-lipophilic balance) value from 1-20, including all ranges and subranges therebetween, with HLB values ranging from 1 to 5 (particularly 3 to 5) or from 15-20 (particularly 16 to 18) being preferred. The alkoxylated fatty alcohol may be chosen from ethoxylated fatty alcohols, propoxylated fatty alcohols, and mixtures thereof.

The alkoxylated fatty alcohol can be chosen from di-alkyl, tri-alkyl- and combinations of di-alkyl and tri-alkyl substituted ethoxylated polymers. They can also be chosen from mono-alkyl, di-alkyl, tri-alkyl, tetra-alkyl substituted alkyl ethoxylated polymers and all combinations thereof. The alkyl group can be saturated or unsaturated, branched or linear and contain a number of carbon atoms preferably from about 12 carbon atoms to about 50 carbon atoms, including all ranges and subranges therebetween, for example, 20 to 40 carbon atoms, 22 to 24 carbon atoms, 30 to 50 carbon atoms, and 40 to 60 carbon atoms. Preferably, the fatty portion contains a mixture of compounds of varying carbon atoms such as, for example, C₂₀-C₄₀ compounds, C₂₂-C₂₄ compounds, C₃₀-C₅₀ compounds, and C₄₀-C₆₀ compounds.

Preferably, the alkoxylated portion of the alkoxylated fatty alcohols of the present disclosure contain 2 or more alkoxylation units, preferably from 2 to 20 alkoxylation units, preferably from 2 to 12 alkoxylation units, preferably from 10 to 200 alkoxylation units, preferably from 20 to 150 alkoxylation units, and preferably from 25 to 100 alkoxylation units, including all ranges and subranges therebetween. Also preferably, the alkoxylation units contain 2 carbon atoms (ethoxylation units) and/or 3 carbon atoms (propoxylation units).

The amount of alkoxylation can also be determined by the percent by weight of the alkoxylated portion with respect to the total weight of the compound. Suitable weight percentages of the alkoxylated portion with respect to the total weight of the compound include, but are not limited to, 10 percent to 95 percent, preferably 20 percent to 90 percent, including all ranges and subranges therebetween with 75 percent to 90 percent (particularly 80 percent to 90 percent) or 20 percent to 50 percent being preferred.

Preferably, the alkoxylated fatty alcohols of the present invention have a number average molecular weight (Mn) greater than 500, preferably from 500 to 5,000, including all ranges and subranges therebetween such as, for example, Mn of 500 to 1250 or an Mn of 2,000 to 5,000.

Suitable examples of alkoxylated fatty alcohols include: laureth-3, laureth-4, laureth-7, laureth-9, laureth-12, laureth-23, ceteth-10, steareth-10, steareth-2, steareth-100, beheneth-5, beheneth-5, beheneth-10, oleth-10, Pareth alcohols, trideceth-10, trideceth-12, C12-13 pareth-3, C12-13 pareth-23, C11-15 pareth-7, PEG hydrogenated castore oil, PEG-75 lanolin, polysorbate-80, polysobate-20, PPG-5 ceteth-20, PEG-55 Propylene Glycol Oleate, glycereth-26 (PEG-26 Glyceryl Ether), PEG 120 methyl glucose dioleate, PEG 120 methyl glucose trioleate, PEG 150 pentaerythrityl tetrastearate, and mixtures thereof.

### (b) Alkoxylated Polyol Esters

The alkoxylated polyol esters may be chosen from pegylated derivatives of propylene glycol oleate, propylene glycol caprylate/caprate, propylene glycol cocoate, propylene glycol stearate, and a mixture thereof. In certain embodiments, the alkoxylated polyol esters are chosen from PEG-55 propylene glycol oleate, PEG-6 propylene glycol caprylate/caprate, PEG-8 propylene glycol cocoate, PEG-25 propylene glycol stearate, and PEG-120 propylene glycol stearate, and a mixture thereof. In some instances, the polyol ester is or includes PEG-55 propylene glycol oleate. While the alkoxylated polyol esters comprise PEG-200 glyceryl stearate in some embodiments, in other embodiments PEG-200 glyceryl stearate may be excluded. Additionally and/or alternatively, the polyol esters may be chosen from ethoxylated fatty acid esters of sorbitan comprising from 2 to 30 mol of ethylene oxide.

In some cases, the polyol ester may be selected from esters of polyols with fatty acids with a saturated or unsaturated chain containing for example from 8 to 24 carbon atoms, preferably 12 to 22 carbon atoms, and alkoxylated derivatives thereof, preferably with a number of alkyleneoxide of from 10 to 200, and more preferably from 10 to 100, such as glyceryl esters of a C₈-C₂₄, preferably C₁₂-C₂₂, fatty acid or acids and alkoxylated derivatives thereof, preferably with a number of alkyleneoxide of from 10 to 200, and more preferably from 10 to 100; polyethylene glycol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sorbitol esters of a C₈-C₂₄, preferably C₁₂-C₂₂, fatty acid or acids and alkoxylated derivatives thereof, preferably with a number of alkyleneoxide of from 10 to 200, and more preferably from 10 to 100; sugar (sucrose, glucose, alkylglycose) esters of a C₈-C₂₄, preferably C₁₂-C₂₂, fatty acid or acids and alkoxylated derivatives thereof, preferably with a number of alkyleneoxide of from 10 to 200, and more preferably from 10 to 100; ethers of fatty alcohols; ethers of sugar and a C₈-C₂₄, preferably C₁₂-C₂₂, fatty alcohol or alcohols; and mixtures thereof.

Examples of ethoxylated fatty esters that may be mentioned include the adducts of ethylene oxide with esters of lauric acid, palmitic acid, stearic acid or behenic acid, and mixtures thereof, especially those containing from 9 to 100 oxyethylene groups, such as PEG-9 to PEG-50 laurate (as the INCI names: PEG-9 laurate to PEG-50 laurate); PEG-9 to PEG-50 palmitate (as the INCI names: PEG-9 palmitate to PEG-50 palmitate); PEG-9 to PEG-50 stearate (as the INCI names: PEG-9 stearate to PEG-50 stearate); PEG-9 to PEG-50 palmitostearate; PEG-9 to PEG-50 behenate (as the INCI names: PEG-9 behenate to PEG-50 behenate); polyethylene glycol 100 EO monostearate (INCI name: PEG-100 stearate); and mixtures thereof.

Sources of unsaturated polyol esters of glycerol include synthesized oils, natural oils (e.g., vegetable oils, algae oils, bacterial derived oils, and animal fats), combinations of these, and the like. Non-limiting examples of vegetable oils include Abyssinian oil, Almond oil, Apricot oil, Apricot Kernel oil, Argan oil, Avocado oil, Babassu oil, Baobab oil, Black Cumin oil, Black Currant oil, Borage oil, Camelina oil, Carinata oil, Canola oil, Castor oil, Cherry Kernel oil, Coconut oil, Corn oil, Cottonseed oil, Echium oil, Evening Primrose oil, Flax Seed oil, Grape Seed oil, Grapefruit Seed oil, Hazelnut oil, Hemp Seed oil, Jatropha oil, Jojoba oil, Kukui Nut oil, Linseed oil, Macadamia Nut oil, Meadowfoam Seed oil, Moringa oil, Neem oil, Olive oil, Palm oil, Palm Kernel oil, Peach Kernel oil, Peanut oil, Pecan oil, Pennycress oil, Perilla Seed oil, Pistachio oil, Pomegranate Seed oil, Pongamia oil, Pumpkin Seed oil, Raspberry oil, Red Palm Olein, Rice Bran oil, Rosehip oil, Safflower oil, Seabuckthorn Fruit oil, Sesame Seed oil, Shea Olein, Sunflower oil, Soybean oil, Tonka Bean oil, Tung oil, Walnut oil, Wheat Germ oil, High Oleoyl Soybean oil, High Oleoyl Sunflower oil, High Oleoyl Safflower oil, High Erucic Acid Rapeseed oil, combinations of these, and the like. Non-limiting examples of animal fats include lard, tallow, chicken fat, yellow grease, fish oil, emu oil, combinations of these, and the like. Non-limiting example of a synthesized oil includes tall oil, which is a byproduct of wood pulp manufacture. In some embodiments, the natural oil is refined, bleached, and/or deodorized.

The polyol esters may optionally be a natural polyol esters chosen from vegetable oil, an animal fat, an algae oil and mixtures thereof; and said synthetic polyol ester is derived from a material selected from the group consisting of ethylene glycol, propylene glycol, glycerol, polyglycerol, polyethylene glycol, polypropylene glycol, poly(tetramethylene ether) glycol, pentaerythritol, dipentaerythritol, tripentaerythritol, trimethylolpropane, neopentyl glycol, a sugar, in one aspect, sucrose, and mixtures thereof.

Additional non-limiting examples of nonionic surfactants that may optionally be used in the cleansing composition include and/or may be chosen from alkanolamides; polyoxyalkylenated nonionic surfactants; polyglycerolated nonionic surfactants; ethoxylated fatty esters; alcohols, alpha-diols, alkylphenols and esters of fatty acids, being ethoxylated, propoxylated or glycerolated; copolymers of ethylene oxide and/or of propylene oxide; condensates of ethylene oxide and/or of propylene oxide with fatty alcohols; polyethoxylated fatty amides; ethoxylated oils from plant origin; fatty acid esters of sucrose; fatty acid esters of polyethylene glycol; N—(C₆-C₂₄)alkylglucamine derivatives, amine oxides such as (C₁₀-C₁₄)alkylamine oxides or N—(C₁₀-C₁₄)acylaminopropylmorpholine oxides; and mixtures thereof.

### (c) Alkoxylated Glycerides

Non-limiting examples of alkoxylated glycerides that may be suitable in certain embodiments include PEG-6 almond glycerides, PEG-20 almond glycerides, PEG-35 almond glycerides, PEG-60 almond glycerides, PEG-192 apricot kernel glycerides, PEG-11 avocado glycerides, PEG-14 avocado glycerides, PEG-11 babassu glycerides, PEG-42 babassu glycerides, PEG-4 caprylic/capric glycerides, PEG-6 caprylic/capric glycerides, PEG-7 caprylic/capric glycerides, PEG-8 caprylic/capric glycerides, PEG-11 cocoa butter glycerides, PEG-75 cocoa butter glycerides, PEG-7 cocoglycerides, PEG-9 cocoglycerides, PEG-20 corn glycerides, PEG-60 corn glycerides, PEG-20 evening primrose glycerides, PEG-60 evening primrose glycerides, PEG-5 hydrogenated corn glycerides, PEG-8 hydrogenated fish glycerides, PEG-20 hydrogenated palm glycerides, PEG-6 hydrogenated palm/palm kernel glyceride, PEG-16 macadamia glycerides, PEG-70 mango glycerides, PEG-13 mink glycerides, PEG-25 moringa glycerides, PEG-42 mushroom glycerides, PEG-2 olive glycerides, PEG-6 olive glycerides, PEG-7 olive glycerides, PEG-10 olive glycerides, PEG-40 olive glycerides, PEG-18 palm glycerides, PEG-12 palm kernel glycerides, PEG-45 palm kernel glycerides, PEG-60 Passiflora edulis seed glycerides, PEG-60 Passiflora incarnata seed glycerides, PEG-45 safflower glycerides, PEG-60 shea butter glycerides, PEG-75 shea butter glycerides, PEG-75 shorea butter glycerides, PEG-35 soy glycerides, PEG-75 soy glycerides, PEG-2 sunflower glycerides, PEG-7 sunflower glycerides, PEG-10 sunflower glycerides, PEG-13 sunflower glycerides, PEG-5 tsubakiate glycerides, PEG-10 tsubakiate glycerides, PEG-20 tsubakiate glycerides, PEG-60 tsubakiate glycerides, and sodium PEG-8 palm glycerides carboxylate.

In some embodiments, the at least one alkoxylated nonionic surfactant includes alkoxylated polyol esters such as polyethylene glycol ethers of esters. For example, the polyethylene glycol ethers of esters may be chosen from PEG-55 propylene glycol oleate, PEG-6 propylene glycol caprylate/caprate, PEG-8 propylene glycol cocoate, PEG-25 propylene glycol stearate, PEG-7 glyceryl cocoate, PEG-30 glyceryl cocoate, laureth-2, laureth-3, laureth-4, PEG-200 glyceryl stearate PEG-55 propylene glycol oleate. In further embodiments, the alkoxylated nonionic surfactants comprise a polyethylene glycol ethers of esters and at least one alkoxylated nonionic surfactant other than a polyethylene glycol ether of an ester.

In an embodiment, the at least one alkoxylated nonionic surfactant comprises at least one polyethylene glycol ether of fatty alcohols. For example, the polyethylene glycol ether of fatty alcohol may be chosen from laureth-2, laureth-3, laureth-4, steareth-20, or a mixtures thereof. The polyethylene glycol ether of fatty alcohols may have from 8 to 30 carbon atoms and in particular from 10 to 22 carbon atoms, such as polyethylene glycol ethers of cetyl alcohol, of stearyl alcohol or of cetearyl alcohol (mixture of cetyl alcohol and stearyl alcohol). Mention may be made, for example, of ethers including from 1 to 200 and preferably from 2 to 100 oxyethylene groups, such as those with the CTFA name Ceteareth-20 or Ceteareth-30, and mixtures thereof.

In an embodiment, the at least one alkoxylated nonionic surfactant comprises at least one polyethylene glycol ether of glycerides. For example, the polyethylene glycol ether of glyceride may be chosen from PEG-6 Caprylic/Capric Glycerides). In another embodiment, the cleansing composition comprises at least two alkoxylated nonionic surfactant. Preferably, one of the at least two alkoxylated nonionic surfactants is PEG-55 propylene glycol oleate.

Further nonionic surfactants that may optionally be present in the cleansing composition include:

### (d) Glucosides

The term glucoside is interchangeable with the term “alkyl polyglucoside.” In some embodiments, the one or more glucosides include those chosen from lauryl glucoside, octyl glucoside, decyl glucoside, coco glucoside, caprylyl/capryl glucoside, sodium lauryl glucose carboxylate, and a mixture thereof. Additionally or alternatively, the glucosides may be a alkyl polyglucoside that is chosen from glycerol (C₆-C₂₄)alkylpolyglycosides including, e.g., polyethoxylated fatty acid mono or diesters of glycerol (C₆-C₂₄)alkylpolyglycosides. Additional alkyl polyglucosides that may be suitably incorporated, in some instances, in the cleansing composition includes alkyl polyglucosides having a structure according to the following formula:

R¹—O—(R²O)ₙ—Z(x)
wherein R¹ is an alkyl group having 8-18 carbon atoms;
R² is an ethylene or propylene group;
Z is a saccharide group with 5 to 6 carbon atoms;
n is an integer from 0 to 10; and
x is an integer from 1 to 5.

Alkyl poly glucosides may, in some instances, include lauryl glucoside, octyl glucoside, decyl glucoside, coco glucoside, caprylyl/capryl glucoside, and sodium lauryl glucose carboxylate. Typically, the at least one alkyl poly glucoside compound is selected from the group consisting of lauryl glucoside, decyl glucoside and coco glucoside. In some instances, decyl glucoside is particularly preferred.

### (e) Alkanolamides

Nonlimiting examples of alkanolamides include fatty acid alkanolamides. The fatty acid alkanolamides may be fatty acid monoalkanolamides or fatty acid dialkanolamides or fatty acid isoalkanolamides, and may have a C₂₋₈ hydroxyalkyl group (the C₂₋₈ chain can be substituted with one or more than one —OH group). Non-limiting examples include fatty acid diethanolamides (DEA) or fatty acid monoethanolamides (MEA), fatty acid monoisopropanolamides (MIPA), fatty acid diisopropanolamides (DIPA), and fatty acid glucamides (acyl glucamides).

Suitable fatty acid alkanolamides may include those formed by reacting an alkanolamine and a C₆-C₃₆ fatty acid. Examples include, but are not limited to: oleic acid diethanolamide, myristic acid monoethanolamide, soya fatty acids diethanolamide, stearic acid ethanolamide, oleic acid monoisopropanolamide, linoleic acid diethanolamide, stearic acid monoethanolamide (Stearamide MEA), behenic acid monoethanolamide, isostearic acid monoisopropanolamide (isostearamide MIPA), erucic acid diethanolamide, ricinoleic acid monoethanolamide, coconut fatty acid monoisopropanolamide (cocoamide MIPA), coconut acid monoethanolamide (Cocamide MEA), palm kernel fatty acid diethanolamide, coconut fatty acid diethanolamide, lauric diethanolamide, polyoxyethylene coconut fatty acid monoethanolamide, coconut fatty acid monoethanolamide, lauric monoethanolamide, lauric acid monoisopropanolamide (lauramide MIPA), myristic acid monoisopropanolamide (Myristamide MIPA), coconut fatty acid diisopropanolamide (cocamide DIPA), and mixtures thereof.

In some instances, the fatty acid alkanolamides preferably include cocamide MIPA, cocamide DEA, cocamide MEA, cocamide DIPA, and mixtures thereof. In particular, the fatty acid alkanolamide may be cocamide MIPA, which is commercially available under the tradename EMPILAN from Innospec Active Chemicals.

Fatty acid alkanolamides include those of the following structure:

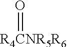

wherein R₄ is an alkyl chain of 4 to 20 carbon atoms (R₄ may be, for example, selected from lauric acid, coconut acid, palmitic acid, myristic acid, behenic acid, babassu fatty acid, isostearic acid, stearic acid, corn fatty acid, soy fatty acid, shea butter fatty acids, caprylic acid, capric acid, and mixtures thereof); wherein R₅ is selected from —CH₂OH, —CH₂CH₂OH, —CH₂CH₂CH₂OH, —CH₂(CHOH)₄CH₂OH, -benzyl, and mixtures thereof; and wherein R₆ is selected from —H, —CH₃, —CH₂OH, —CH₂CH₃, —CH₂CH₂OH, —CH₂CH₂CH₂OH, —CH₂(CHOH)₄CH₂OH, -benzyl, and mixtures thereof.

In some instances, the one or more of the fatty acid alkanolamides include one or more acyl glucamides, e.g., acyl glucamides having a carbon chain length of 8 to 20. Non-limiting examples include lauroyl/myristoyl methyl glucamide, capryloyl/capryl methyl glucamide, lauroyl methyl glucamide, myristoyl methyl glucamide, capryloyl methyl glucamide, capryl methyl glucamide, cocoyl methyl glucamide, capryloyl/caproyl methyl glucamide, cocoyl methyl glucamide, lauryl methylglucamide, oleoyl methylglucamide oleate, stearoyl methylglucamide stearate, sunfloweroyl methylglucamide, and tocopheryl succinate methylglucamide.

### (f) Sorbitan Derivatives

Suitable sorbitan derivatives that may be incorporated into the plurality of nonionic surfactants include those chosen from polysorbate-20 (POE(20) sorbitan monolaurate), polysorbate-21 (POE(4) sorbitan monolaurate), polysorbate-40 (POE(20) sorbitan monopalmitate), polysorbate-60 (POE(20) sorbitan monostearate), polysorbate-61 (POE(4) sorbitan monostearate), polysorbate-65 (POE(20) sorbitan tristearate), polysorbate-80 (POE(20)sorbitan monooleate), polysorbate-81 (POE(4) sorbitan monooleate), polysorbate 85 (POE(20) Sorbitan Trioleate), sorbitan isostearate, sorbitan monolaurate, sorbitan monooleate, sorbitan monopalmitate, sorbitan monostearate, sorbitan sesquioleate, sorbitan trioleate and sorbitan tristearateand a mixture thereof.

Additional and/or alternative sorbitan derivatives include sorbitan esters including, e.g., esters of C₁₆-C₂₂ fatty acid and of sorbitan that were formed by esterification, with sorbitol, of at least one fatty acid comprising at least one saturated or unsaturated linear alkyl chain respectively having from 16 to 22 carbon atoms. These esters can be chosen in particular from sorbitan stearates, behenates, arachidates, palmitates or oleates, and their mixtures. Examples of optional sorbitan esters include sorbitan monostearate (INCI name: Sorbitan stearate) sold by Croda under the name Span 60, the sorbitan tristearate sold by Croda under the name Span 65 V, the sorbitan monopalmitate (INCI name: Sorbitan palmitate) sold by Croda under the name Span 40, the sorbitan monooleate sold by Croda under the name Span 80 V or the sorbitan trioleate sold by Uniqema under the name Span 85 V. A preferable sorbitan ester is sorbitan tristearate.

### Cationic Surfactants

The term “cationic surfactant” as used in the present disclosure is a surfactant that may be positively charged when it is contained in the hair treatment compositions according to the present disclosure. The cationic surfactant may bear one or more positive permanent charges or may contain one or more functional groups that are cationizable in the compositions.

Non-limiting examples of cationic surfactants include cetrimonium chloride, stearimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, and mixtures thereof.

The one or more cationic surfactants may be selected from quaternary ammonium compounds, fatty dialkylamines, or mixtures thereof.

Nonlimiting examples of quaternary ammonium compounds include cetrimonium chloride, steartrimonium chloride, behentrimonium chloride, behentrimonium methosulfate, behenamidopropyltrimonium methosulfate, stearamidopropyltrimonium chloride, arachidtrimonium chloride, distearyldimonium chloride, dicetyldimonium chloride, tricetylmonium chloride, and combinations thereof.

Nonlimiting examples of fatty dialkylamines include oleamidopropyl dimethylamine, linoleamidopropyl dimethylamine, isostearamidopropyl dimethylamine, oleyl hydroxyethyl imidazoline, stearamidopropyl dimethylamine, behenamidopropyl dimethylamine, behenamidopropyl diethylamine, behenamidoethyl diethylamine, behenamidoethyl dimethylamine, arachidamidopropyl dimethylamine, arachidamidopropyl diethylamine, arachidamidoethyl diethylamine, arachidamidoethyl dimethylamine, brassicamidopropyl dimethylamine, lauramidopropyl dimethylamine, myristamidopropyl dimethylamine, dilinoleamidopropyl dimethylamine, palmitamidopropyl dimethylamine, salts thereof, and combinations thereof.

In various embodiments, the one or more cationic surfactants are preferably selected from cetrimonium chloride, behentrimonium chloride, behentrimonium methosulfate, stearamidopropyl dimethylamine, brassicamidopropyl dimethylamine or a mixture thereof.

The total amount of the one or more cationic surfactants added to the initial composition may be from about 0.01 to about 10 wt. %, based on the total weight of the initial composition. In further embodiments, the initial composition includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more cationic surfactants, based on the total weight of the initial composition.

The total amount of the one or more cationic surfactants in the final emulsions will vary but can be from about 0.01 to about 10 wt. %, based on the total weight of the final emulsion. In further embodiments, the emulsions includes from about 0.01 to about 8 wt. %, about 0.01 to about 6 wt. %, about 0.01 to about 5 wt. %, about 0.01 to about 3 wt. %, about 0.1 to about 10 wt. %, about 0.1 to about 8 wt. %, about 0.1 to about 6 wt. %, about 0.1 to about 5 wt. %, about 0.1 to about 3 wt. %, about 0.5 to about 10 wt. %, about 0.5 to about 8 wt. %, about 0.5 to about 6 wt. %, about 0.5 to about 5 wt. %, about 0.5 to about 3 wt. %, about 1 to about 10 wt. %, about 1 to about 8 wt. %, about 1 to about 6 wt. %, about 1 to about 5 wt. %, or about 1 to about 3 wt. % of the one or more cationic surfactants, based on the total weight of the emulsion.

### (d) Water

The total amount of water in the initial composition will vary but is typically from about 5 to about 40 wt. %, based on the total weight of the initial composition. In further embodiments, the total amount of water in the initial composition is from about 5 to about 35 wt. %, about 5 to about 30 wt. %, about 5 to about 25 wt. %, about 10 to about 40 wt. %, about 10 to about 35 wt. %, about 10 to about 30 wt. %, about 10 to about 25 wt. %, about 15 to about 40 wt. %, about 15 to about 35 wt. %, about 15 to about 30 wt. %, about 15 to about 25 wt. %, about 20 to about 40 wt. %, about 20 to about 35 wt. %, about 20 to about 30 wt. %, or about 20 to about 35 wt. %, based on the total weight of the final emulsion.

The total amount of water in the final emulsion will vary but is typically from about 50 to about 95 wt. % based on the total weight of the final emulsion. In further embodiments, the total amount of water in the final emulsion is from about 50 to about 93 wt. %, about 50 to about 90 wt. %, about 50 to about 80 wt. %, about 50 to about 50 wt. %, about 60 to about 99 wt. %, about 60 to about 93 wt. %, about 60 to about 90 wt. %, about 60 to about 90 wt. %, about 70 to about 95 wt. %, about 70 to about 93 wt. %, about 75 to about 95 wt. %, about 75 to about 93 wt. %, about 80 to about 95 wt. %, about 80 to about 93 wt. %, about 85 to about 95 wt. %, or about 85 to about 93 wt. %, based on the total weight of the final emulsion.

In other embodiments, the total amount of water may be from about 35 wt. % to about 80 wt. %, about 40 to about 80 wt. %, about 40 to about 60 wt. %, about 30 to about 60 wt. %, or about 35 to about 50 wt. %, or about 30 to about 50 wt. %, based on the total weight of the final emulsion.

### Water Soluble Solvent

The compositions of the instant disclosure can optionally include one or more water soluble solvents. The term “water soluble solvent” is interchangeable with the terms “water soluble organic solvent” and “water-miscible solvent” and means a compound that is liquid at 25° C. and at atmospheric pressure (760 mmHg), and it has a solubility of at least 50% in water under these conditions. In some cases, the water-soluble solvents have a solubility of at least 60%, 70%, 80%, or 90%. Non-limiting examples of water-soluble solvents include, for example, organic solvents selected from glycerin, mono-alcohols (for example C₂-8, or C₂-4 alcohols), polyols (polyhydric alcohols), glycols, and a mixture thereof.

Nonlimiting examples of water-soluble organic solvents. Non-limiting examples of water-soluble organic solvents include, for example, organic solvents selected from alcohols (for example C₂₋₆ or C₂₋₄ alcohols), polyols (polyhydric alcohols), glycols, and a mixture thereof. Nonlimiting examples of monoalcohols and polyols include ethyl alcohol, isopropyl alcohol, propyl alcohol, benzyl alcohol, and phenylethyl alcohol, or glycols or glycol ethers such as, for example, monomethyl, monoethyl and monobutyl ethers of ethylene glycol, propylene glycol or ethers thereof such as, for example, monomethyl ether of propylene glycol, butylene glycol, hexylene glycol, dipropylene glycol as well as alkyl ethers of diethylene glycol, for example monoethyl ether or monobutyl ether of diethylene glycol. Other suitable examples of organic solvents are ethylene glycol, propylene glycol, butylene glycol, hexylene glycol, and propane diol.

Further non-limiting examples of water soluble organic solvents include alkanediols (polyhydric alcohols) such as 1,2,6-hexanetriol, trimethylolpropane, ethylene glycol, propylene glycol, diethylene glycol, triethylene glycol, tetraethylene glycol, pentaethylene glycol, dipropylene glycol, 2-butene-1,4-diol, 2-ethyl-1,3-hexanediol, 2-methyl-2,4-pentanediol, (caprylyl glycol), 1,2-hexanediol, 1,2-pentanediol, and 4-methyl-1,2-pentanediol; alkyl alcohols having 1 to 4 carbon atoms such as ethanol, methanol, butanol, propanol, and isopropanol; glycol ethers such as ethylene glycol monomethyl ether, ethylene glycol monoethyl ether, ethylene glycol monobutyl ether, ethylene glycol monomethyl ether acetate, diethylene glycol monomethyl ether, diethylene glycol monoethyl ether, diethylene glycol mono-n-propyl ether, ethylene glycol mono-iso-propyl ether, diethylene glycol mono-iso-propyl ether, ethylene glycol mono-n-butyl ether, ethylene glycol mono-t-butyl ether, diethylene glycol mono-t-butyl ether, 1-methyl-1-methoxybutanol, propylene glycol monomethyl ether, propylene glycol monoethyl ether, propylene glycol mono-t-butyl ether, propylene glycol mono-n-propyl ether, propylene glycol mono-iso-propyl ether, dipropylene glycol monomethyl ether, dipropylene glycol monoethyl ether, dipropylene glycol mono-n-propyl ether, and dipropylene glycol mono-iso-propyl ether; 2-pyrrolidone, N-methyl-2-pyrrolidone, 1,3-dimethyl-2-imidazolidinone, formamide, acetamide, dimethyl sulfoxide, sorbit, sorbitan, acetine, diacetine, triacetine, sulfolane, and a mixture thereof.

Polyhydric alcohols are useful. Examples of polyhydric alcohols include ethylene glycol, diethylene glycol, triethylene glycol, propylene glycol, dipropylene glycol, tripropylene glycol, 1,3-butanediol, 2,3-butanediol, 1,4-butanediol, 3-methyl-1,3-butanediol, 1,5-pentanediol, tetraethylene glycol, 1,6-hexanediol, 2-methyl-2,4-pentanediol, polyethylene glycol, 1,2,4-butanetriol, 1,2,6-hexanetriol, and a mixture thereof. Polyol compounds may also be used. Non-limiting examples include the aliphatic diols, such as 2-ethyl-2-methyl-1,3-propanediol, 3,3-dimethyl-1,2-butanediol, 2,2-diethyl-1,3-propanediol, 2-methyl-2-propyl-1,3-propanediol, 2,4-dimethyl-2,4-pentanediol, 2,5-dimethyl-2,5-hexanediol, 5-hexene-1,2-diol, and 2-ethyl-1,3-hexanediol, and a mixture thereof. In a preferred embodiment, the composition include one or more glycols selected from propylene glycol, butylene glycol, pentylene glycol, hexylene glycol, caprylyl glycol, dipropylene glycol, and mixtures thereof.

The total amount of the one or more water soluble solvents in the initial composition, if present, will vary. Nonetheless, the initial composition may include from about 0.1 to about 30 wt. % of one or more water soluble solvents. In further embodiments, the initial composition may include from about 0.1 to about 20 wt. %, about 0.1 to about 15 wt. %, about 0.1 to about 10 wt. %, about 0.1 to about 5 wt. %, about 1 to about 30 wt. %, about 1 to about 30 wt. %, about 1 to about 15 wt. %, about 1 to about 10 wt. %, about 1 to about 5 wt. %, about 5 to about 30 wt. %, about 5 to about 25 wt. %, about 5 to about 20 wt. %, about 10 to about 30 wt. %, about 10 to about 25 wt. %, about 15 to about 30 wt. %, or about 15 to about 25 wt. % of one or more water soluble solvents, based on the total weight of the initial composition.

The total amount of the one or more water soluble solvents in the final emulsion, if present, will vary. Nonetheless, in final emulsion may contain from about 0.01 to about 35 wt. % of one or more water soluble solvents, based on the total weight of the final emulsion. In further embodiments, the final emulsion include from about 0.01 to about 30 wt. %, about 0.01 to about 25 wt. %, about 0.01 to about 20 wt. %, about 0.01 to about 15 wt. %, about 0.01 to about 10 wt. %, about 0.01 to about 8 wt. %, about 0.01 to about 5 wt. %, about 0.5 to about 35 wt. %, about 0.5 to about 30 wt. %, about 0.5 to about 25 wt. %, about 0.5 to about 20 wt. %, about 0.5 to about 15 wt. %, about 0.5 to about 10 wt. %, about 0.5 to about 5 wt. %, about 1 to about 35 wt. %, about 1 to about 30 wt. %, about 1 to about 25 wt. %, about 1 to about 20 wt. %, about 1 to about 15 wt. %, about 1 to about 10 wt. %, about 1 to about 5 wt. %, about 5 to about 35 wt. %, about 5 to about 30 wt. %, about 5 to about 25 wt. %, about 5 to about 20 wt. %, about 5 to about 15 wt. %, about 10 to about 30 wt. %, about 10 to about 25 wt. %, about 15 to about 30 wt. %, or about 20 to about 30 wt. %, based on the total weight of the final emulsion.

### Cationic Conditioning Polymers

The compositions of the instant disclosure may optionally include one or more cationic polymers. Cationic polymers for purposes of the instant disclosure are polymers bearing a positive charge or incorporating cationic entities in their structure. The cationic polymers can comprise mixtures of monomer units derived from amine- and/or quaternary ammonium-substituted monomer and/or compatible spacer monomers. Cationic polymers often provide conditioning benefits to the hair treatment compositions and therefore may be referred to as “cationic conditioning polymers.” Non-limiting examples of cationic polymers include copolymers of 1-vinyl-2-pyrrolidine and 1-vinyl-3-methyl-imidazolium salt (e.g., chloride salt) (referred to as Polyquaternium-16); copolymers of 1-vinyl-2-pyrrolidine and dimethylaminoethyl methacrylate (referred to as Polyquaternium-11); cationic diallyl quaternary ammonium-containing polymer including, for example, dimethyldiallyammonium chloride homopolymer and copolymers of acrylamide and dimethyldiallyammonium chloride (referred to as Polyquaternium-6 and Polyquaternium-7); polysaccharide polymers, such as cationic cellulose derivatives and cationic starch derivatives. Cationic cellulose is available as salts of hydroxyethyl cellulose reacted with trimethyl ammonium substituted epoxide (referred to as Polyquaternium-10). Another type of cationic cellulose includes the polymeric quaternary ammonium salts of hydroxyethyl cellulose reacted with lauryl dimethyl ammonium-substituted epoxide (referred to as Polyquaternium-24). Additionally or alternatively, the cationic conditioning polymers may include or be chosen from cationic guar gum derivatives, such as guar hydroxypropyltrimonium chloride.

Preferred cationic polymers include cationic polysaccharide polymers, such as cationic cellulose, cationic starch, and cationic guar gum. In the context of the instant disclosure, cationic polysaccharide polymers include cationic polysaccharides and polysaccharide derivatives (e.g., derivatized to be cationic), for example, resulting in cationic cellulose (cellulose derivatized to be cationic), cationic starch (derivatized to be cationic), or cationic guar (guar derivatized to be cationic).

Nonlimiting examples of cationic celluloses include hydroxyethylcellulose (also known as HEC), hydroxymethylcellulose, methylhydroxyethylcellulose, hydroxypropylcellulose (also known as HPC), hydroxybutylcellulose, hydroxyethylmethylcellulose (also known as methyl hydroxyethylcellulose) and hydroxypropylmethylcellulose (also known as HPMC), cetyl hydroxyethylcellulose, polyquaternium-10, polyquaternium-24, and mixtures thereof, preferably polyquaternium-10, polyquaternium-24, and mixtures thereof.

Nonlimiting examples of cationic guar include guar hydroxypropyltrimonium chloride, hydroxypropyl guar hydroxypropyltrimonium chloride, guar hydroxypropyltrimethylammonium chloride, and mixtures thereof.

Nonlimiting examples of cationic starch include starch hydroxypropyltrimonium chloride, hydroxypropyl oxidized starch PG trimonium chloride, and a mixture thereof.

In certain embodiments, the composition may include one or more polyquaterniums. Nonlimiting examples include polyquaternium-1, polyquaternium-2, polyquaternium-3, polyquaternium-4, polyquaternium-5, polyquaternium-6, polyquaternium-7, polyquaternium-8, polyquaternium-9, polyquaternium-10, polyquaternium-11, polyquaternium-12, polyquaternium-13, polyquaternium-14, polyquaternium-15, polyquaternium-16, polyquaternium-17, polyquaternium-18, polyquaternium-19, polyquaternium-20, polyquaternium-21, polyquaternium-22, polyquaternium-23, polyquaternium-24, polyquaternium-25, polyquaternium-26, polyquaternium-27, polyquaternium-28, polyquaternium-29, polyquaternium-30, polyquaternium-40, polyquaternium-41, polyquaternium-42, polyquaternium-43, polyquaternium-44, polyquaternium-45, polyquaternium-46, polyquaternium-47, polyquaternium-48, polyquaternium-49, polyquaternium-50, polyquaternium-51, polyquaternium-52, polyquaternium-53, polyquaternium-54, polyquaternium-55, polyquaternium-56, polyquaternium-57, polyquaternium-58, polyquaternium-59, polyquaternium-60, polyquaternium-61, polyquaternium-62, polyquaternium-63, polyquaternium-64, polyquaternium-65, polyquaternium-66, polyquaternium-67, etc. In some cases, preferred polyquaternium compounds include polyquaternium-10, polyquaternium-11, polyquaternium-67, and a mixture thereof.

In certain embodiments, the composition may include polyquaternium-1 (ethanol, 2,2′,2″-nitrilotris-, polymer with 1,4-dichloro-2-butene and N,N,N′,N′-tetramethyl-2-butene-1,4-diamine), polyquaternium-2, (poly[bis(2-chloroethyl) ether-alt-1,3-bis[3-(dimethylamino)propyl]urea]), polyquaternium-4, (hydroxyethyl cellulose dimethyl diallylammonium chloride copolymer; Diallyldimethylammonium chloride-hydroxyethyl cellulose copolymer), polyquaternium-5 (copolymer of acrylamide and quaternized dimethylammoniumethyl methacrylate), polyquaternium-6 (poly(diallyldimethylammonium chloride)), polyquaternium-7 (copolymer of acrylamide and diallyldimethylammonium chloride), polyquaternium-8 (copolymer of methyl and stearyl dimethylaminoethyl ester of methacrylic acid, quaternized with dimethylsulphate), polyquaternium-9 (homopolymer of N,N-(dimethylamino)ethyl ester of methacrylic acid, quaternized with bromomethane), polyquaternium-10 (quaternized hydroxyethyl cellulose), polyquaternium-11 (copolymer of vinylpyrrolidone and quaternized dimethylaminoethyl methacrylate), polyquaternium-12 (ethyl methacrylate/abietyl methacrylate/diethylaminoethyl methacrylate copolymer quaternized with dimethyl sulfate), polyquaternium-13 (ethyl methacrylate/oleyl methacrylate/diethylaminoethyl methacrylate copolymer quaternized with dimethyl sulfate), polyquaternium-14 (trimethylaminoethylmethacrylate homopolymer), polyquaternium-15 (acrylamide-dimethylaminoethyl methacrylate methyl chloride copolymer), Polyquaternium-16 (copolymer of vinylpyrrolidone and quaternized vinylimidazole), Polyquaternium-17 (adipic acid, dimethylaminopropylamine and dichloroethylether copolymer), Polyquaternium-18 (azelanic acid, dimethylaminopropylamine and dichloroethylether copolymer), polyquaternium-19 (copolymer of polyvinyl alcohol and 2,3-epoxypropylamine), polyquaternium-20 (copolymer of polyvinyl octadecyl ether and 2,3-epoxypropylamine), polyquaternium-22 (copolymer of acrylic acid and diallyldimethylammonium chloride), polyquaternium-24 (auaternary ammonium salt of hydroxyethyl cellulose reacted with a lauryl dimethyl ammonium substituted epoxide), polyquaternium-27 (block copolymer of Polyquaternium-2 and Polyquaternium-17), polyquaternium-28 (copolymer of vinylpyrrolidone and methacrylamidopropyl trimethylammonium), polyquaternium-29 (chitosan modified with propylen oxide and quaternized with epichlorhydrin), polyquaternium-30 (ethanaminium, N-(carboxymethyl)-N,N-dimethyl-2-[(2-methyl-1-oxo-2-propen-1-yl)oxy]-, inner salt, polymer with methyl 2-methyl-2-propenoate), polyquaternium-31 (N,N-dimethylaminopropyl-N-acrylamidine quatemized with diethylsulfate bound to a block of polyacrylonitrile), polyquaternium-32 (poly(acrylamide 2-methacryloxyethyltrimethyl ammonium chloride)), polyquaternium-33 (copolymer of trimethylaminoethylacrylate salt and acrylamide), polyquaternium-34 (copolymer of 1,3-dibromopropane and N,N-diethyl-N′,N′-dimethyl-1,3-propanediamine), Polyquaternium-35 (methosulphate of the copolymer of methacryloyloxyethyltrimethylammonium and of methacryloyloxyethyldimethylacetylammonium), polyquaternium-36 (copolymer of N,N-dimethylaminoethylmethacrylate and buthylmethacrylate, quaternized with dimethylsulphate), polyquaternium-37 (poly(2-methacryloxyethyltrimethylammonium chloride)), polyquaternium-39 (terpolymer of acrylic acid, acrylamide and diallyldimethylammonium Chloride), polyquaternium-42 (poly[oxyethylene(dimethylimino)ethylene (dimethylimino)ethylene dichloride]), Polyquaternium-43 (copolymer of acrylamide, acrylamidopropyltrimonium chloride, 2-amidopropylacrylamide sulfonate and dimethylaminopropylamine), polyquaternium-44 (3-Methyl-1-vinylimidazolium methyl sulfate-N-vinylpyrrolidone copolymer), polyquaternium-45 (copolymer of (N-methyl-N-ethoxyglycine)methacrylate and N,N-dimethylaminoethylmethacrylate, quaternized with dimethyl sulphate), polyquaternium-46 (terpolymer of vinylcaprolactam, vinylpyrrolidone, and quaternized vinylimidazole), polyquaternium-47 (terpolymer of acrylic acid, methacrylamidopropyl trimethylammonium chloride, and methyl acrylate), and/or polyquaternium-67.

In certain embodiments, the compositions of the instant disclosure include one or more cationic polymers selected from cationic cellulose derivatives, quaternized hydroxyethyl cellulose (e.g., polyquaternium-10), cationic starch derivatives, cationic guar gum derivatives, copolymers of acrylamide and dimethyldiallyammonium chloride (e.g., polyquaternium-7), polyquaterniums, and a mixture thereof. For example, the cationic polymer(s) may be selected from polyquaterniums, for example, polyquaterniums selected from polyquaternium-4, polyquaternium-5, polyquaternium-6, polyquaternium-7, polyquaternium-10, polyquaternium-22, polyquaternium-37, polyquaternium-39, polyquaternium-47, polyquaternium-53, polyquaternium-67 and a mixture thereof. A combination of two or more polyquaterniums can be useful. A particularly preferred and useful cationic polymer is polyquaternium-10.

In certain embodiments, the compositions include one or more cationic polymers chosen from cationic proteins and cationic protein hydrolysates (e.g., hydroxypropyltrimonium hydrolyzed wheat protein), quaternary diammonium polymers (e.g., hexadimethrine chloride), copolymers of acrylamide and dimethyldiallyammonium chloride, and mixtures thereof.

The total amount of the one or more cationic polymers in the initial composition, if present, will vary but can be in an amount from about 0.1 to about 10 wt. %, based on the total weight of the initial composition. In further embodiments, the initial composition can include from about 0.1 to about 8 wt. %, about 0.1 to about 5 wt. %, about 1 to about 10 wt. %, about 1 to about 5 wt. %, about 1 to about 3 wt. %, based on the total weight of the initial composition.

The total amount of the one or more cationic polymers in the final composition, if present, will vary but can be in an amount from about 0.01 to about 5 wt. %, based on the total weight of the final composition. In further embodiments, the final composition can include from about 0.01 to about 4 wt. %, about 0.01 to about 3 wt. %, about 0.01 to about 2 wt. %, about 0.05 to about 5 wt. %, about 0.05 to about 4 wt. %, about 0.05 to about 3 wt. %, about 0.05 to about 2 wt. %, about 0.1 to about 5 wt. %, about 0.1 to about 4 wt. %, about 0.1 to about 3 wt. %, about 0.1 to about 2 wt. %, about 0.1 to about 1.5 wt. %, about 0.2 to about 5 wt. %, about 0.2 to about 4 wt. %, about 0.2 to about 3 wt. %, about 0.2 to about 2 wt. %, about 0.2 to about 1.5 wt. % of the one or more cationic polymers, based on the total weight of the final composition.

### Film-Forming Polymer

In various embodiments, the compositions of the instant disclosure optionally include one or more film-forming polymers. Nonlimiting examples of film-forming polymers include polyurethanes, vinyl polymers, natural polymers, latex polymers, vinylpyrrolidone (VP)-based polymers, amphoteric polymers, and mixtures thereof. The film forming polymers may be added into the initial composition or may be added into the final composition, for example, depending on the hydrophobic/hydrophilic properties of the film forming polymer.

The total amount of the one or more film-forming polymers, if present, will vary. In certain embodiments, the final emulsion can include from about 0.01 to about 10 wt. % of one or more film-forming polymers, based on the total weight of final emulsion. In further embodiments, the final emulsion can include from about 0.01 to about 5 wt. %, about 0.01 to about 3 wt. %, about 0.1 to about 10 wt. %, about 0.1 to about 5 wt. %, about 0.1 to about 3 wt. %, about 0.5 to about 10 wt. %, about 0.5 to about 8 wt. %, about 0.5 to about 5 wt. %, about 0.5 to about 3 wt. %, about 1 to about 10 wt. %, about 1 to about 8 wt. %, about 1 to about 5 wt. %, or about 1 to about 3 wt. %, based on the total weight of the final emulsion.

### (i) Polyurethanes

The polyurethanes may be aliphatic, cycloaliphatic, or aromatic polyurethanes, polyurea-urethanes or polyurea copolymers, comprising, alone or as a mixture: at least one block of aliphatic and/or cycloaliphatic and/or aromatic polyester origin, and/or at least one branched or unbranched silicone block, for example polydimethylsiloxane or polymethylphenylsiloxane, and/or at least one block comprising fluoro groups.

The film-forming polyurethanes that can be used in the invention may also be obtained from branched or non-branched polyesters or from alkyls comprising labile hydrogens, which are modified by reaction with a diisocyanate and a difunctional organic compound (for example dihydroxy, diamino or hydroxyamino), also comprising either a carboxylic acid or carboxylate group, or a sulfonic acid or sulfonate group, or alternatively a neutralizable tertiary amine group or a quaternary ammonium group.

With a view to forming the polyurethane, monomers bearing an anionic group that can be used during the polycondensation that maybe mentioned include dimethylolpropionic acid, trimellitic acid or a derivative such as trimellitic anhydride, the sodium salt of 3-sulfopentanediol acid, and the sodium salt of 5-sulfo-1,3-benzenedicarboxylic acid. Preferably, the monomer bearing an anionic group is dimethylolpropionic acid.

As film-forming polyurethane that may be used according to the invention, mention may thus be made of the aqueous polyurethane dispersions sold under the names AVALURE UR-405®, AVALURE UR-410®, AVALURE UR-425® and AVALURE UR-450® by the company Goodrich. A particularly preferred polyurethane is polyurethane-99.

Advantageously, the film-forming polyurethanes that are selected from copolymers obtained by copolymerization of hexanediol, neopentyl glycol, adipic acid, hexamethylene diisocyanate, N-(2-aminoethyl)-3-aminoethanesulfonic acid and ethylenediamine. Preferably, the polyurethanes may also be selected from copolymers obtained by copolymerization of adipic acid, dicyclohexylmethane diisocyanate, ethylenediamine, hexanediol, neopentyl glycol and sodium N-(2-aminoethyl)-3-aminoethanesulfonate.

In particular, the polyurethanes are chosen from those sold under the name BAYCUSAN ECO E 1001, BAYCUSAN C1001 or C1004, known as polyurethane-99, polyurethane-35, and more particularly the product sold under the name BAYCUSAN C1001, known as polyurethane-99.

### (ii) Vinyl Polymers

Vinyl polymers may be chosen from polyvinyl alcohols, copolymers derived from C₄-C₈ monounsaturated carboxylic acids or anhydrides, and methyl vinyl ether/butyl monomaleate copolymers. For the purpose of the present invention, the term “polyvinyl alcohol” means a polymer comprising —CH₂CH(OH)— units. The polyvinyl alcohols are generally produced by hydrolysis of polyvinyl acetate. Usually, the reaction takes place in the presence of methanol (alcoholysis). The reaction is normally catalyzed by acidic or basic catalysis. The degree of hydrolysis of the commercial products is variable, often around 87%, but products with a 100 degree of hydrolysis also exist. Copolymers with monomers other than vinyl acetate also exist, such as ethylene/vinyl alcohol copolymers.

The polyvinyl alcohol polymers are preferably chosen from homopolymers or copolymers with vinyl acetate, the latter corresponding in particular to a partial hydrolysis of polyvinyl acetate.

Use may, for example, be made of the products of the CELVOL range provided by the company Celanese under the names CELVOL 540, CELVOL 350, CELVOL 325, CELVOL 165, CELVOL 125, CELVOL 540 S, CELVOL 840 and CELVOL 443.

The copolymer(s) derived from C₄-C₈ monounsaturated carboxylic acids or anhydrides may be chosen from copolymers comprising (i) one or more maleic, fumaric or itaconic acids or anhydrides and (ii) one or more monomers chosen from vinyl esters, vinyl ethers, vinyl halides, phenylvinyl derivatives, and acrylic acid and its esters, the anhydride functions of these copolymers being optionally monoesterified or monoamidated, for example, INCI name: Butyl Ester of PVM/MA Copolymer. Preferably, the copolymer(s) derived from C₄-C₈ monounsaturated carboxylic acids or anhydrides are chosen from the monoesterified methyl vinyl ether/maleic anhydride copolymers, for example, ethyl ester of PVM/MA copolymer, sold under the name GANTREZ ES 225 by the company ISP.

(iii) Natural Polymers

The polymers may also be chosen from natural polymers, in particular polysaccharides which have monosaccharides or disaccharides as base units. The natural polymers are preferably chosen from pullulan, guar gums and modified guar gums, celluloses, and gellan gum, and derivatives thereof.

In a preferred embodiment, a natural film-forming polymer is pullulan. Pullulan is a polysaccharide polymer consisting of maltotriose units, also known as α-1,4-; α-1,6-glucan′. Three glucose units in maltotriose are connected by an α-1,4 glycosidic bond, whereas consecutive maltotriose units are connected to each other by an α-1,6 glycosidic bond. In various embodiments, the skin perfecting compositions include pullulan and optionally one or more additional film-forming polymers, for example, one or more polyurethanes. In such situation, the amount of the pullulan will vary.

Guar gums are galactomannans consisting of mannose and galactose. For the purpose of the present disclosure, the term “modified guar gum” means guar gums alkylated with at least one C₁-C₈ alkyl group, guar gums hydroxyalkylated with at least one C₁₋₈ hydroxyalkyl group and guar gums acylated with at least one C₁₋₈ acyl group. Hydroxypropylated guar gums (e.g., hydroxypropyl guar) such as the product sold under the name JAGUAR HP 105 by the company Rhodia is a useful example.

The cellulose is a β1-4-polyacetal of cellobiose, cellobiose being a disaccharide consisting of two glucose molecules. The cellulose derivatives may be cationic, amphoteric or nonionic. Among these derivatives, cellulose ethers, cellulose esters and cellulose ester ethers are distinguished. Among the nonionic cellulose ethers, mention may be made of alkylcelluloses such as methylcelluloses and ethylcelluloses; hydroxyalkylcelluloses such as hydroxymethylcelluloses, hydroxyethylcelluloses and hydroxypropylcelluloses; and mixed hydroxyalkyl-alkylcelluloses such as hydroxypropylmethylcelluloses, hydroxyethylmethylcelluloses, hydroxyethylethylcelluloses and hydroxybutylmethylcelluloses.

Among the cationic cellulose ethers, mention may be made of crosslinked or non-crosslinked quaternized hydroxyethylcelluloses. The quaternizing agent may especially be glycidyltrimethylammonium chloride or a fatty amine such as laurylamine or stearylamine. Another cationic cellulose ether that may be mentioned is hydroxyethylcellulosehydroxypropyltrimethylammonium. Among the cellulose esters are mineral esters of cellulose (cellulose nitrates, sulfates, phosphates, etc.), organic cellulose esters (cellulose monoacetates, triacetates, amidopropionates, acetatebutyrates, acetatepropionates and acetatetrimellitates, etc.), and mixed organic/mineral esters of cellulose, such as cellulose acetatebutyrate sulfates and cellulose acetatepropionate sulfates.

Among the cellulose ester ethers, mention may be made of hydroxypropylmethylcellulose phthalates and ethylcellulose sulfates. The cellulose-based compounds of the invention may be chosen from unsubstituted celluloses and substituted celluloses.

The celluloses and derivatives are represented, for example, by the products sold under the names AVICEL® (microcrystalline cellulose, MCC) by the company FMC Biopolymers, under the name METHOCEL™ (cellulose ethers) and ETHOCEL™ (ethylcellulose) by the company Dow, BENECEL® (methylcellulose), BLANOSE™ (carboxymethylcellulose), CULMINAI® (methylcellulose, hydroxypropylmethylcellulose), KLUCEL® (hydroxypropylcellulose), POLYSURF® (cetylhydroxyethylcellulose) and NATROSOL® CS (hydroxyethylcellulose) by the company Hercules Aqualon.

Gellan gum is a polysaccharide produced by aerobic fermentation of Sphingomonas elodea, more commonly known as Pseudomonas elodea. This linear polysaccharide is formed from the sequence of the following monosaccharides: D-glucose, D-glucuronic acid and L-rhamnose. In native form, gellan gum is highly acylated. The gellan gum preferably used in the film according to the present invention is a gellan gum that is at least partially deacylated. This at least partially deacylated gellan gum is obtained by high-temperature alkaline treatment. A solution of KOH or of NaOH will, for example, be used. The purified gellan gum sold under the trade name KELCOGEL® by the company Kelco is suitable for preparing the compositions according to the invention.

Gellan gum derivatives are all the products obtained by performing standard chemical reactions, especially such as esterifications, addition of a salt of an organic or mineral acid. Welan gum is used, for example, as a gellan gum derivative. Welan gum is a gellan gum modified by fermentation by means of Alcaligenes strain ATCC 31 555. Welan gum has a recurring pentasaccharide structure formed from a main chain consisting of D-glucose, D-glucuronic acid and L-rhamnose units, onto which a pendent L-rhamnose or L-mannose unit is grafted. The welan gum (diutan gum) sold under the trade name KELCO CRETE® by the company Kelco is suitable for preparing the compositions according to the invention.

As other saccharide polymers that can be used according to the invention, mention may be made of starches and derivatives thereof.

Natural film-forming polymers include celluloses and derivatives thereof, in particular those sold under the name AVICEL® (microcrystalline cellulose, MCC) by the company FMC Biopolymers.

Carrageenans are anionic polysaccharides constituting the cell walls of various red algae (Rhodophyceae) belonging to the Gigartinacae, Hypneaceae, Furcellariaceae and Polyideaceae families. They are generally obtained by hot aqueous extraction from natural strains of said algae. These linear polymers, formed by disaccharide units, are composed of two D-galactopyranose units linked alternately by a(1,3) and β(1,4) bonds. They are highly sulfated polysaccharides (20%-50%) and the α-D-galactopyranosyl residues may be in 3,6-anhydro form. Depending on the number and position of sulfate-ester groups on the repeating disaccharide of the molecule, several types of carrageenans are distinguished, namely: kappa-carrageenans, which bear one sulfate-ester group, iota-carrageenans, which bear two sulfate-ester groups, and lambda-carrageenans, which bear three sulfate-ester groups. Carrageenans are composed essentially of potassium, sodium, magnesium, triethanolamine and/or calcium salts of polysaccharide sulfate esters.

Carrageenans are sold especially by the company SEPPIC under the name SOLAGUM®, by the company Gelymar under the names CARRAGEL®, CARRALACT® and Carrasol®, by the company Cargill under the names Satiagel™ and SATIAGUM™, and by the company CP-Kelco under the names GENULACTA®, GENUGEL® and GENUVISCO®.

Mention is made of hyaluronic acid and salts therefore, for example sodium hyaluronate and potassium hyaluronate. Sodium hyaluronate is the sodium salt of hyaluronic acid. It is a glycosaminoglycan and long-chain polymer of disaccharide units of Na-glucuronate-N-acetylglucosamine.

A useful film forming polymer is xanthan gum and modified xanthan gums, such as dehydroxanthan gum, hydroxypropyl xanthan gum, and mixtures thereof. In some cases, dehydroxanthan gum is useful.

### (iv) Latex or Pseudolatex Polymers

As presented above for the film-forming polyurethanes, the film-forming polymer may thus also be present in a composition of the invention in the form of particles dispersed in an aqueous phase, which is generally known as a latex or pseudolatex. Techniques for preparing these dispersions are well known to those skilled in the art.

Aqueous dispersions of film-forming polymers that may be used include the acrylic dispersions sold under the names NEOCRYL XK-90, NEOCRYL A-1070®, NEOCRYL A-1090®, NEOCRYL BT-62®, NEOCRYL A-1079® and NEOCRYL A-523® by the company Avecia-Neoresins, Dow Latex 432® by the company Dow Chemical, DAITOSOL 5000 AD® or DAITOSOL 5000 SJ® by the company Daito Kasey Kogyo; SYNTRAN 5760® or SYNTRAN PC 5100® by the company Interpolymer, Allianz OPT by the company Röhm & Haas, aqueous dispersions of acrylic or styrene/acrylic polymers sold under the brand name JONCRYL® by the company Johnson Polymer, or the aqueous dispersions of polyurethane sold under the names NEOREZ R-981® and NEOREZ R-974® by the company Avecia-Neoresins, AVALURE UR-405®, AVALURE UR-410®, AVALURE UR-425®, AVALURE UR-450®, SANCURE 875®, SANCURE 861®, SANCURE 878® and SANCURE 2060® by the company Goodrich, IMPRANIL 85® by the company Bayer and AQUAMERE H-1511® by the company Hydromer; the sulfopolyesters sold under the brand name EASTMAN AQ® by the company Eastman Chemical Products, and vinyl dispersions, for instance MEXOMER PAM® from the company Chimex, and mixtures thereof.

### (v) Vinylpyrrolidone (VP)-Based Polymers

Nonlimiting examples of vinylpyrrolidone (VP)-based film-forming polymers include polyvinylpyrrolidone (PVP), VP-styrene copolymer, VP-vinyl acetate copolymer, and diethyl sulfate VP-dimethylaminoethyl-methacrylic acid copolymer, and blends thereof.

### (vi) Amphoteric Polymers

Nonlimiting examples of film-forming polymers include amphoteric polymers. Nonlimiting examples include polymethacryloyloxyethyltrimethyl ammonium chloride, alkyl vinyl ether maleic anhydride (AVE/MA) copolymer, poly-2-aminopropyl acrylate, poly(diethylaminoethyl methacrylate), copolymers such as dimethylaminoethyl methacrylate copolymer and zwitterionic polymers such as polybetaines such as poly-2-ethynyl-N-(4-sulfobutyl)pyridinium betaine (PESPB), polysulfobetaines such as poly-N,N-dimethyl-N-3-sulfopropyl-3′methacrylamidopropanaminium and copolymers such as diallyldimethylammonium chloride-maleamic acid copolymers, or a combination thereof.

### Miscellaneous Ingredients

The compositions and emulsions of the instant disclosure optionally include one or more miscellaneous ingredients. Miscellaneous ingredients are ingredients that are compatible with the compositions and emulsions and do not disrupt or materially affect the basic and novel properties. Nonlimiting examples of ingredients include preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, etc. In various embodiments, the miscellaneous ingredients are chosen from preservatives, fragrances, pH adjusters, salts, chelating agents, buffers, composition colorants, and mixtures thereof. In the context of the instant disclosure, a “composition colorant” is a compound that colors the composition but does not have an appreciable coloring effect on hair. In other words, the composition colorant is included to provide a coloring to the composition for aesthetic appeal but is not intended to impart coloring properties to hair. Styling gels, for example, can be found in a variety of different colors (e.g., light blue, light pink, etc.) yet application of the styling gel to hair does not visibly change the color of the hair.

The total amount of the one or more miscellaneous ingredients in the initial composition, if present, will vary. Nonetheless, in various embodiments, the initial composition can include about 0.1 to about 15 wt. % of the one or more miscellaneous ingredients, based on the total weight of the compositions. In further embodiments, the initial composition includes about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the initial composition.

The total amount of the one or more miscellaneous ingredients in the final emulsion, if present, will vary. Nonetheless, in various embodiments, the final emulsion includes about 0.1 to about 15 wt. % of the one or more miscellaneous ingredients, based on the total weight of the compositions. In further embodiments, the final emulsion includes about 0.1 to about 12 wt. %, about 0.1 to about 10 wt. %, about 0.1 to about 5 wt. %, about 0.5 to about 15 wt. %, about 0.5 to about 12 wt. %, about 0.5 to about 10 wt. %, about 0.5 to about 8 wt. %, about 0.5 to about 5 wt. %, about 1 to about 15 wt. %, about 1 to about 12 wt. %, about 1 to about 10 wt. %, about 1 to about 8 wt. %, about 1 to about 5 wt. %, about 2 to about 15 wt. %, about 2 to about 12 wt. %, about 2 to about 10 wt. %, about 2 to about 8 wt. %, or about 2 to about 5 wt. %, based on the total weight of the final emulsion.

Droplet Size In various embodiments, the average droplet size is from about 10 nm to about 2 μm, about 10 nm to about 1.5 μm, about 10 nm to about 1 μm, about 10 nm to about 800 nm, about 10 nm to about 600 nm, about 10 nm to about 500 nm, about 10 nm to about 250 nm, about 50 nm to about 2 μm, about 50 nm to about 1.5 μm, about 50 nm to about 1 μm, about 50 nm to about 800 nm, about 50 nm to about 600 nm, about 50 nm to about 500 nm, about 50 nm to about 250 nm, about 100 nm to about 2 μm, about 100 nm to about 1.5 μm, about 100 nm to about 1 μm, about 100 nm to about 800 nm, about 100 nm to about 600 nm, about 100 nm to about 500 nm, about 100 nm to about 300 nm, about 150 nm to about 500 nm, about 150 nm to about 400 nm, about 200 nm to about 500 nm, or about 200 nm to about 300 nm.

Droplet size can be determined using Brookhaven Dynamic Light Scattering (DLS). DLS is a technique used to determine droplet size in a colloidal system or emulsion. When the samples are illuminated with a monochromatic laser beam, the particles in the samples undergo Brownian motion, causing fluctuations in scattered light intensity. The scattered light is then collected at various angles, and the autocorrelation function of these intensity fluctuations is analyzed. The analysis provides information about the rate of diffusion of the particles, and from this, the size distribution is inferred using mathematical models. Brookhaven's DLS instruments use advanced algorithms to accurately interpret the data, offering insights into the dynamic behavior and size characteristics of particles ranging from a few nanometers to several micrometers in a liquid medium.

pH

The pH of the compositions and emulsions will vary. Nonetheless, the compositions and emulsions typically have a pH from about 4.5 to about 8.5. In further embodiments, the pH of the compositions and emulsions is from about 4.5 to about 8, about 4.5 to about 7.5, about 4.5 to about 7, about 4.5 to about 6.5, about 4.5 to about 6, about 5 to about 8.5, about 5 to about 8, about 5 to about 7.5, about 5 to about 7, about 5 to about 6.5, about 5 to about 6, about 5.5 to about 8.5, about 5.5 to about 8, about 5.5 to about 7.5, about 5.5 to about 7, about 5.5 to about 6.5, about 6 to about 8.5, about 6 to about 8, about 6 to about 7.5, or about 6 to about 7.

### EMBODIMENTS

In preferred embodiments, the emulsions (the cosmetic or personal care composition) have a small oil phase and comprise, consist of, or consist essentially of:
(a) about 0.1 to about 8 wt. %, preferably about 0.5 to about 5 wt. %, more preferably about 1 to about 5 wt. % of a hydrophobic polymer, which is a reaction product of a natural or food-derived oil and a methacrylate or acrylate polymer, wherein preferably,
   the hydrophobic polymer is a reaction product of: (a)(i) a natural or food-derived oil selected from linseed oil, sunflower oil, tung oil, fish oil, cottonseed oil, soybean oil, or combinations thereof, preferably linseed oil and (a)(ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
   the hydrophobic polymer is the reaction product of linseed oil and poly(isobutyl methacrylate);
(b) about 0.1 to about 10 wt. %, preferably about 0.1 to about 6 wt. %, more preferably about 0.5 to about 5 wt. % of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}, wherein more preferably, the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably, at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C₁₂-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 0.1 to about 15 wt. %, preferably about 0.5 to about 10 wt. %, more preferably about 1 to about 8 wt. % of one or more surfactants, wherein the surfactants are selected from:
   (c)(i) one or more biosurfactants, preferably wherein at least one of the one or more biosurfactants is a glycolipid selected from sophorolipids, rhamnolipids, trehalose lipids, mannosylerythritol lipids, and combinations thereof, wherein preferably at least one of the one or more biosurfactants is a rhamnolipid; and/or
   (c)(ii) one or more additional surfactants other than the one or more biosurfactants of (c)(i), preferably wherein the one or more additional surfactants other than the biosurfactants of (c)(i) include one or more anionic surfactants, one or more nonionic surfactants, one or more amphoteric surfactants, or combination thereof;
(d) about 75 to about 95 wt. %, preferably about 80 to about 95 wt. %, more preferably about 85 to about 93 wt. % of water;
(e) optionally, one or more water soluble solvents, preferably wherein the one or more water soluble solvents, if present, is selected from mono-alcohols (for example C₂₋₈, or C₂₋₄ alcohols), polyols (polyhydric alcohols), glycols, and a mixture thereof, wherein if present, the one or more water soluble solvents is in an amount of about 0.01 to about 20 wt. %, preferably about 0.1 to about 15 wt. %, more preferably about 0.5 to about 10 wt. %; and
(f) optionally, one or more film forming polymers; and
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion, and
   all percentages by weight are based on the total weight of the emulsion.

The pH of the emulsion may be from about 4.5 to about 8.5. In further embodiments, the pH of the emulsion may be from about 5 to about 8, about 5 to about 7, about 5 to less than 7, about 5.5 to about 6.5, or about 6 to about 7.5.

The average droplet size of the oil droplets in the emulsion by be from about 10 nm to about 2 μm. The average droplet size may preferably be from about 50 nm to about 1 μm, more preferably from about 100 nm to about 500 nm.

In preferred embodiments, the emulsions (the cosmetic or personal care composition) have a large oil phase and comprise, consist of, or consist essentially of:
(a) about 0.1 to about 8 wt. %, preferably about 0.5 to about 5 wt. %, more preferably about 1 to about 5 wt. % of a hydrophobic polymer, which is a reaction product of a natural or food-derived oil and a methacrylate or acrylate polymer, wherein preferably,
   the hydrophobic polymer is a reaction product of: (a)(i) a natural or food-derived oil selected from linseed oil, sunflower oil, tung oil, fish oil, cottonseed oil, soybean oil, or combinations thereof, preferably linseed oil, and (a)(ii) a polymer derived from monomers selected from isobutyl methacrylate, methyl methacrylate, ethyl methacrylate, n-butyl methacrylate, and combinations thereof, preferably isobutyl methacrylate polymer, wherein even more preferably,
   the hydrophobic polymer is the reaction product of linseed oil and poly(isobutyl methacrylate);
(b) about 1 to about 50 wt. %, more preferably about 10 to about 45 wt. %, even more preferably about 10 to about 30 wt. %, and even more preferably about 15 to about 30 wt. % of one or more solvents capable of solubilizing the reaction product of (a), wherein preferably,
   the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.8 MPa^{0.5},
P₁ is 4.8 MPa^{0.5}, and
H₁ is 13.0 MPa^{0.5}, wherein more preferably,
the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (1):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably,
at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof;
(c) about 0.1 to about 15 wt. %, preferably about 0.5 to about 10 wt. %, more preferably about 1 to about 8 wt. % of one or more surfactants, wherein the surfactants are preferably selected from:
   (c)(i) one or more biosurfactants, preferably wherein at least one of the one or more biosurfactants is a glycolipid selected from sophorolipids, rhamnolipids, trehalose lipids, mannosylerythritol lipids, and combinations thereof, wherein preferably at least one of the one or more biosurfactants is a rhamnolipid; and/or
   (c)(ii) one or more additional surfactants other than the one or more biosurfactants of (c)(i), preferably wherein the one or more additional surfactants other than the biosurfactants of (c)(i) include one or more anionic surfactants, one or more nonionic surfactants, one or more amphoteric surfactants, or combination thereof;
(d) about 40 to about 95 wt. %, preferably about 50 to about 90 wt. %, more preferably about 60 to about 85 wt. % of water;
(e) optionally, one or more water soluble solvents, preferably wherein the one or more water soluble solvents, if present, is selected from mono-alcohols (for example C₂₋₈, or C₂₋₄ alcohols), polyols (polyhydric alcohols), glycols, and a mixture thereof, wherein if present, the one or more water soluble solvents is in an amount of about 0.1 to about 30 wt. %, preferably about 0.5 to about 20 wt. %, more preferably about 1 to about 10 wt. %; and
(f) optionally, one or more film forming polymers; and
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion, and all percentages by weight are based on the total weight of the emulsion.

The pH of the emulsion may be from about 4.5 to about 8.5. In further embodiments, the pH of the emulsion may be from about 5 to about 8, about 5 to about 7, about 5 to less than 7, about 5.5 to about 6.5, or about 6 to about 7.5.

The average droplet size of the oil droplets in the emulsion by be from about 10 nm to about 2 μm. The average droplet size may preferably be from about 50 nm to about 1 μm, more preferably from about 100 nm to about 500 nm.

In another preferred embodiments, the emulsions (the cosmetic or personal care composition) comprise, consist of, or consist essentially of:
(a) about 0.1 to about 8 wt. %, preferably about 0.5 to about 5 wt. %, more preferably about 1 to about 5 wt. % of a hydrophobic polymer which is the reaction product of linseed oil and poly(isobutyl methacrylate);
(b) about 0.1 to about 10 wt. %, preferably about 0.1 to about 6 wt. %, more preferably about 0.5 to about 5 wt. % of one or more solvents capable of solubilizing the reaction product of (a), wherein the one or more solvents capable of solubilizing the reaction product of (a) have a dispersion component (D), a polar component (P), and a hydrogen bonding component (H), and a distance (Ra) of less than or equal to 9.9 MPa^{0.5} per Hansen Solubility Parameters, wherein the distance (Ra) is defined by formula (I):

$$Ra = \sqrt{4 {( D - {D}_{1} )}^{2} + {( P - {P}_{1} )}^{2} + {( H - {H}_{1} )}^{2}} \qquad ( I )$$

wherein
D₁ is 16.4 MPa^{0.5},
P₁ is 5.0 MPa^{0.5}, and
H₁ is 11.7 MPa^{0.5}, wherein eve more preferably, at least one of the one or more solvents capable of dissolving the hydrophobic polymer of (a) are selected from dioctylcyclohexane, mineral oil, isocetyl palmitate, isocetyl palmitate, cyclopentasiloxane, dicaprylyl carbonate, octyl isostearate, trimethylhexyl isononanoate, 2-ethylhexyl isononanoate, dicaprylyl ether, dihexyl carbonate, polydecene, octyl cocoate, isodecyl neopentanoate, isohexy decanoate, isodecyl octanoate, dihexyl ether, isododecane, isodecyl 3,5,5 trimethyl hexanoate, oleyl erucate, Passiflora incarnata oil, jojoba oil, octyl palmitate, macadamia nut oil, isopropyl stearate, rapeseed oil, hexyl decanol, isotridecyl 3,5,5 trimethylhexanonanoate, polycitronellol acetate, mixed decanoyl and octanoyl glycerides, 2-ethylhexanoic acid, 3,5,5 trimethyl ester, cetystearyl octanoate, dimethicone, isopropyl palmitate, octyldodecanol, dioctyl adipate, isopropyl myristate, octyl palmitate (2-ethylhexyl palmitate), octyldodeceyl myristate, butyl octanoic acid, isopropyl stearate, caprylic/capric triglycerides, isopropyl isostearate, Jojoba oil, cyclomethicone, groundnut oil, almond oil, sunflower oil, decyl oleate, avocado oil, olive oil, dibutyl adipate, castor oil, calendula oil, wheatgerm oil, decyl oleate, avocado oil, calendula oil, propylene glycol monoisostearate, cocoglycerides, butylene glycol caprylate/caprate, C12-15 alkyl benzoate, caprylic/capric diglyceryl succinate, caprylic/capric triglyceride, cetearyl isonoanoate, cetearyl octanoate, cetyl dimethicone, coco-caprylate/caprate, cocoglycerides, Di-C12-13 alkyl tartrate, dibutyl adipate, dicaprylyl carbonate, dicaprylyl ether, hexyl decanol, hydrogenated polyisobutene, isoeicosane, isohexadecane, isopropyl palmitate, isopropyl stearate, octyl cocoate, octyl isostearate, octyl octanoate, octyl palmitate, octyl stearate, octyl dodecanol, octyldodecyl myristate, isopropyl stearate, pentaerythrityl tetraisostearate, phenyl trimethicone, polydecene, propylene glycol dicaprylate/dicaprate, stearyl heptanoate, tricaprylin, tridecyl stearate, tridecyl trimellitate, triisostearin, or combinations thereof, even more preferably wherein at least one of the one or more solvents capable of solubilizing the hydrophobic polymer of (a) is selected from caprylic/capric triglyceride, polycitronellol acetate, isododecane, and combinations thereof;
(c) about 0.1 to about 15 wt. %, preferably about 0.5 to about 10 wt. %, more preferably about 1 to about 8 wt. % of one or more surfactants, preferably a plurality of surfactants, wherein preferably the plurality of surfactants include:
   (c)(i) about 0.1 to about 10 wt. %, preferably about 0.5 to about 8 wt. %, more preferably about 1 to about 5 wt. % of one or more biosurfactants, preferably wherein at least one of the one or more biosurfactants is a glycolipid selected from sophorolipids, rhamnolipids, trehalose lipids, mannosylerythritol lipids, and combinations thereof, wherein preferably at least one of the one or more biosurfactants is a rhamnolipid; and
   (c)(ii) about 0.1 to about 8 wt. %, preferably about 0.5 to about 6 wt. %, more preferably about 1 to about 4 wt. % of one or more additional surfactants other than the one or more biosurfactants, preferably wherein the one or more additional surfactants other than the biosurfactants of (c)(i) is one or more anionic surfactants, one or more nonionic surfactants, one or more amphoteric surfactants or combination thereof; wherein more preferably,
      the one or more surfactants other than the one or more biosurfactants of (c)(i) are selected from alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof, wherein even more preferably,
      at least one of the one or more anionic surfactants is selected from an acyl taurate, for example, acyl taurates selected from sodium cocoyl taurate, sodium methyl cocoyl taurate, sodium lauroyl taurate, sodium methyl lauroyl taurate, or mixtures thereof;
(d) about 75 to about 95 wt. %, preferably about 80 to about 95 wt. %, more preferably about 85 to about 93 wt. % of water;
(e) optionally, one or more water soluble solvents, preferably wherein the one or more water soluble solvents, if present, is selected from mono-alcohols (for example C₂₋₈, or C₂₋₄ alcohols), polyols (polyhydric alcohols), glycols, and a mixture thereof, wherein if present, the one or more water soluble solvents is in an amount of about 0.01 to about 20 wt. %, preferably about 0.1 to about 15 wt. %, more preferably about 0.5 to about 10 wt. %; and
(f) optionally, one or more film forming polymers, wherein if present, the one or more film forming polymers is preferably in an amount of about 0.1 to about 6 wt. %, more preferably about 0.5 to about 5 wt. %, even more preferably about 0.5 to about 3 wt. %; and
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion, and
   all percentages by weight are based on the total weight of the emulsion.

The pH of the emulsion may be from about 4.5 to about 8.5. In further embodiments, the pH of the emulsion may be from about 5 to about 8, about 5 to about 7, about 5 to less than 7, about 5.5 to about 6.5, or about 6 to about 7.5.

The average droplet size of the oil droplets in the emulsion by be from about 10 nm to about 2 μm. The average droplet size may preferably be from about 50 nm to about 1 μm, more preferably from about 100 nm to about 500 nm.

In further preferred embodiments, the emulsions (the cosmetic or personal care composition) comprise, consist of, or consist essentially of:
(a) about 0.1 to about 8 wt. %, preferably about 0.5 to about 5 wt. %, more preferably about 1 to about 5 wt. % of a hydrophobic polymer which is the reaction product of linseed oil and poly(isobutyl methacrylate);
(b) about 0.1 to about 10 wt. %, preferably about 0.1 to about 6 wt. %, more preferably about 0.5 to about 5 wt. % of one or more solvents capable of solubilizing the reaction product of (a);
(c) about 0.1 to about 15 wt. %, preferably about 0.5 to about 10 wt. %, more preferably about 1 to about 8 wt. % of a plurality of surfactants, wherein the plurality of surfactants include:
   (c)(i) about 0.1 to about 10 wt. %, preferably about 0.5 to about 8 wt. %, more preferably about 1 to about 5 wt. % of one or more rhamnolipids; and
   (c)(ii) about 0.1 to about 8 wt. %, preferably about 0.5 to about 6 wt. %, more preferably about 1 to about 4 wt. % of one or more additional surfactants other than the one or more biosurfactants, preferably wherein the one or more additional surfactants are selected from one or more anionic surfactants, one or more nonionic surfactants, one or more amphoteric surfactants or combination thereof, wherein more preferably,
      the one or more surfactants other than the one or more biosurfactants of (c)(ii) are selected from alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof, wherein even more preferably,
      at least one of the one or more anionic surfactants is selected from an acyl taurates, for example, acyl taurates selected from sodium cocoyl taurate, sodium methyl cocoyl taurate, sodium lauroyl taurate, sodium methyl lauroyl taurate, or mixtures thereof;
(d) about 75 to about 95 wt. %, preferably about 80 to about 95 wt. %, more preferably about 85 to about 93 wt. % of water;
(e) optionally, one or more water soluble solvents, preferably wherein the one or more water soluble solvents, if present, is selected from mono-alcohols (for example C₂₋₈, or C₂₋₄ alcohols), polyols (polyhydric alcohols), glycols, and a mixture thereof, wherein if present, the one or more water soluble solvents is in an amount of about 0.01 to about 20 wt. %, preferably about 0.1 to about 15 wt. %, more preferably about 0.5 to about 10 wt. %; and
(f) optionally, one or more film forming polymers, wherein if present, the one or more film forming polymers is preferably in an amount of about 0.1 to about 6 wt. %, more preferably about 0.5 to about 5 wt. %, even more preferably about 0.5 to about 3 wt. %; and
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion, and
   all percentages by weight are based on the total weight of the emulsion.

The pH of the emulsion may be from about 4.5 to about 8.5. In further embodiments, the pH of the emulsion may be from about 5 to about 8, about 5 to about 7, about 5 to less than 7, about 5.5 to about 6.5, or about 6 to about 7.5.

The average droplet size of the oil droplets in the emulsion by be from about 10 nm to about 2 μm. The average droplet size may preferably be from about 50 nm to about 1 μm, more preferably from about 100 nm to about 500 nm.

In yet another preferred embodiment, the emulsions (the cosmetic or personal care composition) include a larger oil phase and comprise, consist of, or consist essentially of:
(a) about 0.1 to about 8 wt. %, preferably about 0.5 to about 5 wt. %, more preferably about 1 to about 5 wt. % of a hydrophobic polymer which is the reaction product of linseed oil and poly(isobutyl methacrylate);
(b) about 1 to about 50 wt. %, preferably about 5 to about 45 wt. %, more preferably about 10 to about 40 wt. %, and even more preferably about 20 to about 35 wt. % of one or more solvents capable of solubilizing the reaction product of (a);
(c) about 0.1 to about 15 wt. %, preferably about 0.5 to about 10 wt. %, more preferably about 1 to about 8 wt. % of a plurality of surfactants, wherein the plurality of surfactants include:
   (c)(i) about 0.1 to about 10 wt. %, preferably about 0.5 to about 8 wt. %, more preferably about 1 to about 5 wt. % of one or more rhamnolipids; and
   (c)(ii) about 0.1 to about 8 wt. %, preferably about 0.5 to about 6 wt. %, more preferably about 1 to about 4 wt. % of one or more additional surfactants other than the one or more biosurfactants, preferably wherein the one or more additional surfactants are selected from one or more anionic surfactants, one or more nonionic surfactants, one or more amphoteric surfactants or combination thereof, wherein more preferably,
      the one or more surfactants other than the one or more biosurfactants of (c)(ii) are selected from alkyl sulfonates, alkyl sulfosuccinates, alkyl sulfoacetates, acyl isethionates, alkoxylated monoacids, acyl amino acids such as acyl taurates, acyl glycinates, acyl glutamates, acyl sarcosinates, salts thereof, or mixtures thereof, wherein even more preferably,
      at least one of the one or more anionic surfactants is selected from an acyl taurates, for example, acyl taurates selected from sodium cocoyl taurate, sodium methyl cocoyl taurate, sodium lauroyl taurate, sodium methyl lauroyl taurate, or mixtures thereof;
(d) about 40 to about 95 wt. %, preferably about 50 to about 90 wt. %, even more preferably about 60 to about 80 wt. % of water;
(e) optionally, one or more water soluble solvents, preferably wherein the one or more water soluble solvents, if present, is selected from mono-alcohols (for example C₂₋₈, or C₂₋₄ alcohols), polyols (polyhydric alcohols), glycols, and a mixture thereof, wherein if present, the one or more water soluble solvents is in an amount of about 0.01 to about 20 wt. %, preferably about 0.1 to about 15 wt. %, more preferably about 0.5 to about 10 wt. %; and
(f) optionally, one or more film forming polymers, wherein if present, the one or more film forming polymers is preferably in an amount of about 0.1 to about 6 wt. %, more preferably about 0.5 to about 5 wt. %, even more preferably about 0.5 to about 3 wt. %; and
(g) optionally, one or more miscellaneous ingredients, preferably wherein the one or more miscellaneous ingredients, if present, are selected from preservatives, fragrances, pH adjusters, salts, chelating agents, buffers, antioxidants, flavonoids, vitamins, botanical extracts, UV filtering agents, proteins, protein hydrolysates, and/or isolates, fillers (e.g., organic and/or inorganic fillers such as talc, calcium carbonate, silica, etc.) composition colorants, wherein if present, are preferably in an amount of about 0.1 to about 15 wt. %, more preferably about 0.5 to about 10 wt. %, more preferably about 1 to about 6 wt. %;
   wherein the composition is an oil-in-water emulsion, and all percentages by weight are based on the total weight of the emulsion.

The pH of the emulsion may be from about 4.5 to about 8.5. In further embodiments, the pH of the emulsion may be from about 5 to about 8, about 5 to about 7, about 5 to less than 7, about 5.5 to about 6.5, or about 6 to about 7.5.

The average droplet size of the oil droplets in the emulsion by be from about 10 nm to about 2 μm. The average droplet size may preferably be from about 50 nm to about 1 μm, more preferably from about 100 nm to about 500 nm.

Implementation of the present disclosure is provided by way of the following examples. The examples serve to illustrate the technology without being limiting in nature.

### EXAMPLES

Implementation of the present disclosure is provided by way of the following examples. The following examples serve to elucidate aspects of the technology without being limiting in nature.

### Example 1

### Standard Preparation Process

Three emulsions (A, B, and C) were prepared according to a standard procedure known in the art for preparing emulsion. The ingredients were directly combined, without first solubilizing the Mycelx® in solvent and surfactants. The compositions were processed with a high-speed Silverson Homogenizer capable of generating fine droplet sizes in the range of 2-5 microns. Despite subjecting the compositions to the high-speed homogenization process, the droplet size of the resulting emulsions was greater than 1 micron, the compositions were not transparent, and the compositions phase separated at 45° C.

Standard Emulsions

| | | A | B | C |
|---|---|---|---|---|
| (a) | MYCELX ®¹ | 2.6 | 2.6 | 2.6 |
| (b) | m-RHAMNOLIPID | 3.2 | 3.2 | 3.2 |
| | SODIUM METHYL | 1.3 | 1.3 | 1.3 |
| | COCOYL TAURATE | | | |
| (c) | CAPRYLIC/CAPRIC | 0.6 | 0 | 0 |
| | TRIGLYCERIDE | | | |
| | POLYCITRONELLOL | 0 | 0.6 | 0 |
| | ACETATE | | | |
| | ISODODECANE | 0 | 0 | 0.6 |
| (d) | WATER | QS | QS | QS |

| | | | |
|---|---|---|---|
| DROPLETS SIZE | >4 μm | >4 μm | >4 μm |
| BY DILUTION | | | |
| 1:100 [nm] | | | |
| Translucent | No | No | No |
| Stable 45° C. for 2 Month | No | No | No |

¹Reaction product of linseed oil and isobutyl methacrylate polymer

After forming the emulsions, the compositions were further diluted by a 1:100 ratio with water and the droplet size measured using Brookhaven Dynamic Light Scattering (DLS). DLS is a technique used to determine droplet size in a colloidal system or emulsion. A Brookhaven Instrument was used to determine the size distribution of particles in each sample. When the samples are illuminated with a monochromatic laser beam, the particles in the samples undergo Brownian motion, causing fluctuations in scattered light intensity. The scattered light is then collected at various angles, and the autocorrelation function of these intensity fluctuations was analyzed. The analysis provides information about the rate of diffusion of the particles, and from this, the size distribution is inferred using mathematical models. Brookhaven's DLS instruments use advanced algorithms to accurately interpret the data, offering insights into the dynamic behavior and size characteristics of particles ranging from a few nanometers to several micrometers in a liquid medium.

### Example 2

### Inventive Preparation Process

Three initial compositions (D(i), E(i), and F(i)) were prepared by mixing the m-rhamnolipid, sodium methyl cocoyl taurate and MycelX®, solvent (caprylic/capric triglyceride, polycitronellol acetate, isododecane), and water in the amounts shown in the table below at 2,500 rpm for 2 minutes at 25° C. The compositions were homogenous.

Initial Compositions

| | | D(i) | E(i) | F(i) |
|---|---|---|---|---|
| (a) | MYCELX ®¹ | 25.5 | 25.8 | 25.8 |
| (b) | m-RHAMNOLIPID | 31.6 | 31.6 | 31.6 |
| | SODIUM METHYL | 13.1 | 13.1 | 13.1 |
| | COCOYL TAURATE | | | |
| (c) | CAPRYLIC/CAPRIC | 6.5 | 0 | 0 |
| | TRIGLYCERIDE | | | |
| | POLYCITRONELLOL | 0 | 6.5 | 0 |
| | ACETATE | | | |
| | ISODODECANE | 0 | 0 | 6.5 |
| (d) | WATER | QS | QS | QS |

¹Reaction product of linseed oil and isobutyl methacrylate polymer

### Example 3

### Inventive Process and Compositions

The initial compositions of Example 2 ((D(i), E(i), and F(i), were further processed to generate dispersions (D, E, and F). The initial compositions from Example 2 were diluted with water and gently mixed to generate dispersions. The dispersions form without requiring high-speed mixers or other energy intensive procedure. The gentle mixing used to generate the dispersions was simple shaking of the compositions to uniformly mix the base compositions with additional water.

Oil-In-Water Dispersion

| | | D | E | F |
|---|---|---|---|---|
| (a) | MYCELX ®¹ | 2.6 | 2.6 | 2.6 |
| (b) | m-RHAMNOLIPID | 3.2 | 3.2 | 3.2 |
| | SODIUM METHYL | 1.3 | 1.3 | 1.3 |
| | COCOYL TAURATE | | | |
| (c) | CAPRYLIC/CAPRIC | 0.6 | 0 | 0 |
| | TRIGLYCERIDE | | | |
| | POLYCITRONELLOL | 0 | 0.6 | 0 |
| | ACETATE | | | |
| | ISODODECANE | 0 | 0 | 0.6 |
| (d) | WATER | QS | QS | QS |

| | | | |
|---|---|---|---|
| DROPLETS SIZE | 260 | 229 | 228 |
| BY DILUTION | | | |
| 1:100 [nm] | | | |
| Translucent | Yes | Yes | Yes |
| Stable 45° C. for 2 Months | Yes | Yes | Yes |

¹Reaction product of linseed oil and isobutyl methacrylate polymer

After forming the dispersions (D, E, and F), they were further diluted by a 1:100 ratio with water and the droplet size measured using Brookhaven Dynamic Light Scattering (DLS). DLS is a technique used to determine droplet size in a colloidal system or emulsion. A Brookhaven Instrument was used to determine the size distribution of particles in each sample. When the samples are illuminated with a monochromatic laser beam, the particles in the samples undergo Brownian motion, causing fluctuations in scattered light intensity. The scattered light is then collected at various angles, and the autocorrelation function of these intensity fluctuations was analyzed. The analysis provides information about the rate of diffusion of the particles, and from this, the size distribution is inferred using mathematical models. Brookhaven's DLS instruments use advanced algorithms to accurately interpret the data, offering insights into the dynamic behavior and size characteristics of particles ranging from a few nanometers to several micrometers in a liquid medium.

### Example 4

### Interfacial Tension

Studies were carried out to determine how MycelX® contributes to the interfacial tension of the oil phase using a drop shape analysis with a Biolab Optical Tensiometer. The procedure involves capturing and analyzing the shape of a liquid droplet formed at the interface between two immiscible fluids. The Biolab Optical Tensiometer utilizes advanced optical techniques to precisely capture the droplet shape, and the interfacial tension is calculated based on the geometrical parameters of the droplet. In the process of interfacial tension measurement using drop shape analysis with a Biolab Optical Tensiometer, a small droplet of one liquid (Myclex® and oil) is dispensed onto the surface of another immiscible liquid (water and surfactant or only water), forming an interface. The Biolab Optical Tensiometer captures high-resolution images of the droplet and the system's software analyzes the droplet's contour with accuracy. This involves extracting key geometrical parameters, such as the droplet diameter and height. The interfacial tension is then calculated using the Young-Laplace equation, which correlates the pressure difference across the curved interface with the droplet's shape and interfacial tension. This method provides a non-invasive and efficient means of determining interfacial tension, offering valuable insights into the surface properties of liquids, and aiding in the characterization of materials across diverse industries. The results are presented in the table below.

Wt. %

Oil Phase

| | | | | | | |
|---|---|---|---|---|---|---|
| MYCELX ®¹ | 0 | 95 | 90 | 0 | 83 | 50 |
| POLYCITRONELLOL ACETATE | 100 | 5 | 10 | 0 | 0 | 0 |
| CAPRYLIC/CAPRIC | 0 | 0 | 0 | 100 | 17 | 50 |
| TRIGLYCERIDE | | | | | | |

Water Phase with 0.005% m-Rhamnolipid

| | | | | | | |
|---|---|---|---|---|---|---|
| Interfacial Tension [mN/m] | 3.5 | ~1 | ~1 | 2.32 | ~1 | 1.48 |

Water Phase Without m-Rhamnolipid

| | | | | | | |
|---|---|---|---|---|---|---|
| Interfacial Tension [mN/m] | 17 | 10 | 10 | 21 | 12 | 32 |

¹Reaction product of linseed oil and isobutyl methacrylate polymer.

The results indicate that the MycelX® contributes to an ultralow interfacial tension resulting in formation of the dispersions with very small droplet sizes. This is apparent based on the data in Example 1, which shows that the surfactants alone are not capable of producing the dispersions. The MycelX® interacts with the surfactants and reduces interfacial tension allowing for smaller droplet sizes. The lower the interfacial tension, the smaller the dispersed droplet size. Interfacial tension is like a “tug-of-war” between interphase of oil phase and the aqueous phase. It is the force that keeps the two phases from easily mixing. The unit for interfacial tension is usually measured in millinewtons per meter (mN/m) or dynes per centimeter (dyn/cm). The higher the interfacial tension, the less likely an oil phase and aqueous phase will easily blend, resulting in bigger droplet sizes, and less stable emulsions.

### Example 4

### Foam Boosting Properties

Testing was carried out to determine how MycelX® influences foam generation and durability. The emulsions set forth in the table below were prepared according to the procedure described in Example 2, i.e., initial compositions were first prepared and subsequently diluted to form the emulsions.

| | Inventive | | | Comp. |
|---|---|---|---|---|
| INGREDIENTS | 1 | 2 | 3 | 4 |
| MYCELX ®¹ | 0.6 | 0.6 | 5.2 | 0 |
| Rhamnolipid | 0.7 | 0.7 | 5.7 | 0.7 |
| SODIUM METHYL | 0.3 | 0.3 | 2.3 | 0.3 |
| COCOYL TAURATE | | | | |
| POLYCITRONELLOL | | 0.1 | | |
| ACETATE | | | | |
| CAPRYLIC/CAPRIC | 0.1 | | | 0.1 |
| TRIGLYCERIDE | | | | |
| Initial Foam | 59.4 | 47.0 | 56.2 | 31.1 |
| Volume (mL) | | | | |
| Final Foam | 47.8 | 38.0 | 40.1 | 26.6 |
| Volume (mL) | | | | |

¹Reaction product of linseed oil and isobutyl methacrylate polymer.

A Kruiss Dynamic Foam Analyzer (DFA100) was used to measure the foamability of the compositions at a temperature of 25° C. The device uses an accurately controlled foaming process and an optical sensor that measure the quantity (volume) of foam produced and the decay characteristic of the foam, i.e., the lastingness of the foam. The results reported in the table above show that both initial foam volume and final foam volume was significantly greater for the inventive compositions containing MycelX®.

The data show that in addition to the surprising stability and reduction of droplet size provided by MycelX®, MycelX® also provides an unexpected improvement with respect to foaming generation and longevity.

The foregoing description illustrates and describes the disclosure. Additionally, the disclosure shows and describes only the preferred embodiments. However, as mentioned above, it is to be understood that it is capable to use in various other combinations, modifications, and environments and is capable of changes or modifications within the scope of the invention concepts as expressed herein, commensurate with the above teachings and/or the skill or knowledge of the relevant art. The embodiments described herein above are further intended to explain best modes known by applicant and to enable others skilled in the art to utilize the disclosure in such, or other, embodiments and with the various modifications required by the particular applications or uses thereof. Accordingly, the description is not intended to limit the invention to the form disclosed herein. Also, it is intended to the appended claims be construed to include alternative embodiments.

As used herein, the terms “comprising,” “having,” and “including” are used in their open, non-limiting sense.

The terms “a,” “an,” and “the” are understood to encompass the plural as well as the singular. Thus, the term “a mixture thereof” also relates to “mixtures thereof.” Throughout the disclosure, the term “a mixture thereof” is used, following a list of elements as shown in the following example where letters A-F represent the elements: “one or more elements selected from the group consisting of A, B, C, D, E, F, and a mixture thereof.” The term, “a mixture thereof” does not require that the mixture include all of A, B, C, D, E, and F (although all of A, B, C, D, E, and F may be included). Rather, it indicates that a mixture of any two or more of A, B, C, D, E, and F can be included. In other words, it is equivalent to the phrase “one or more elements selected from the group consisting of A, B, C, D, E, F, and a mixture of any two or more of A, B, C, D, E, and F.”

Likewise, the term “a salt thereof” also relates to “salts thereof.” Thus, where the disclosure refers to “an element selected from the group consisting of A, B, C, D, E, F, a salt thereof, and a mixture thereof,” it indicates that that one or more of A, B, C, D, and F may be included, one or more of a salt of A, a salt of B, a salt of C, a salt of D, a salt of E, and a salt of F may be included, or a mixture of any two of A, B, C, D, E, F, a salt of A, a salt of B, a salt of C, a salt of D, a salt of E, and a salt of F may be included.

The salts referred to throughout the disclosure may include salts having a counter-ion such as an alkali metal, alkaline earth metal, or ammonium counterion. This list of counterions, however, is non-limiting. Appropriate counterions for the components described herein are known in the art.

The expression “one or more” means “at least one” and thus includes individual components as well as mixtures/combinations.

The term “plurality” means “more than one” or “two or more.”

The term “transparent” with respect to a transparent composition indicates that the composition has transmittance of at least 80% at a wavelength of 600 nm, for example measured using a Lambda 40 UV-visible spectrometer. The compositions may have, for example, a transmittance of at least 80%, at least 90%, or at least 95% at a wavelength of 600 nm, measured, for example, using a Lambda 40 UV-visible spectrometer. The term “clear” is interchangeable with the term “transparent” for purposes of the instant disclosure.

The term “translucent” with respect to a translucent composition indicates that the composition has a transmittance of at least 50% at a wavelength of 600 nm, for example measured using a Lambda 40 UV-visible spectrometer.

Other than in the operating examples, or where otherwise indicated, all amount expressing quantities of ingredients and/or reaction conditions may be modified in all instances by the term “about,” meaning within +/−5% of the indicated number. Thus, for a range of “about 1 to about 10 wt. %,” The lower amount of “about 1 wt. %” may extend down to 0.95 wt. %, which is 5% less than 1 wt. %. The higher amount of “about 10 wt. %” may extend up to 10.5 wt. %, which is 5% higher than 10 wt. %, i.e., a range of “0.95 wt. % to 10.5 wt. %.”

All percentages, parts and ratios herein are based upon the total weight of the compositions of the present invention, unless otherwise indicated.

Some of the various categories of components identified may overlap. In such cases where overlap may exist and the composition includes both components (or the composition includes more than two components that overlap), an overlapping compound does not represent more than one component. For example, certain compounds may be considered both oily solvent and a surfactant. If a particular composition includes both an oily solvent and a surfactant, a single compound will serve as only the oily solvent or only as the surfactant (the single compound does not simultaneously serve as both the oily solvent and the surfactant).

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

The term “substantially free” or “essentially free” as used herein means that there is less than about 2% by weight of a specific material added to a composition, based on the total weight of the compositions. Nonetheless, the compositions may include less than about 1 wt. %, less than about 0.5 wt. %, less than about 0.1 wt. %, or none of the specified material. For example, if a composition is essentially free from compound X, the composition includes less that 2 wt. % of compound X, or less than 1 wt. % of compound X, or less than 0.5 wt. % of compound X, or less than 0.1 wt. % of compound X, or is free from compound X.

All components that are positively set forth in the instant disclosure may be negatively excluded from the claims, e.g., a claimed composition may be “free,” “essentially free” (or “substantially free”) of one or more components that are positively set forth in the instant disclosure.

All publications and patent applications cited in this specification are herein incorporated by reference in their entirety, and for any and all purposes, as if each individual publication or patent application were specifically and individually indicated to be incorporated by reference. In the event of an inconsistency between the present disclosure and any publications or patent application incorporated herein by reference, the present disclosure controls.

1. A cosmetic composition comprising:
(a) a hydrophobic polymer formed as a reaction product of a natural or food-derived oil and a methacrylate or acrylate polymer;
(b) one or more solvents capable of solubilizing the hydrophobic polymer of (a);
(c) one or more surfactants; and
(d) water;
wherein the composition is an oil-in-water emulsion.

2. The composition of claim 1, wherein the hydrophobic polymer is the reaction product of a natural or food-derived oil and a methacrylate polymer.

3. The composition of claim 1, wherein the natural or food-derived oil is a drying oil or semi-drying oil selected from linseed oil, sunflower oil, tung oil, fish oil, cottonseed oil, soybean oil, or combinations thereof.

4. The composition of claim 1, wherein the polymer is derived from monomers selected from isobutyl methacrylate, methyl methacrylate, ethyl methacrylate, n-butyl methacrylate, and combinations thereof.

5. The composition of claim 1, wherein the hydrophobic polymer is the reaction product of about 50 to about 85 parts by weight of the natural or food-derived oil and about 15 to about 50 parts by weight of the methacrylate or acrylate polymer.

6. The composition of claim 1, wherein the hydrophobic polymer is the reaction product of about 72 to about 77 parts by weight of the natural or food-derived oil and about 23 to about 28 parts by weight of a methacrylate polymer.

7. The composition of claim 1, wherein the polymer is isobutyl methacrylate polymer.

8. The composition of claim 1, wherein the hydrophobic polymer is the reaction product of linseed oil and poly(isobutyl methacrylate).

9. The composition of claim 1, wherein the one or more solvents capable of solubilizing the reaction product of (a) have a dispersion component (D), a polar component (P), and a hydrogen bonding component (H), and a distance (Ra) of less than or equal to 13.4 MPa^{0.5} per Hansen Solubility Parameters, wherein the distance (Ra) is defined by formula (I):
$$Ra = \sqrt{4 {( D - {D}_{1} )}^{2} + {( P - {P}_{1} )}^{2} + {( H - {H}_{1} )}^{2}} \qquad ( I )$$
wherein
D₁ is 16.8 MPa^{0.5},
P₁ is 4.8 MPa^{0.5}, and
H₁ is 13.0 MPa^{0.5}.

10. The composition of claim 1, wherein at least one of the one or more solvents capable of solubilizing the reaction product of (a) is selected from polycitronellol acetate, caprylic/capric triglyceride, isododecane, isohexadecane, tetradecane, isopropyl myristate, isopropyl alcohol, octyldodecanol, ethanol, phenoxyethanol, castor oil, and mixtures thereof.

11. The composition of claim 1, wherein at least one of the one or more surfactants is a biosurfactant.

12. The composition of claim 11, wherein the biosurfactant is a glycolipid.

13. The composition of claim 12, wherein the glycolipid is selected from sophorolipids, rhamnolipids, trehalose lipids, mannosylerythritol lipids, and combinations thereof.

14. The composition of claim 13, wherein the glycolipid is a rhamnolipid.

15. The composition of claim 1, wherein at least one of the one or more surfactants is selected from anionic surfactants, nonionic surfactants, amphoteric surfactants, or combinations thereof.

16. The composition of claim 15 comprising an anionic surfactant selected from sulfate surfactants, glutamate surfactants, acyl taurates, alkanoyl isethionates, alkyl succinates, alkyl sulphosuccinates, N-alkoyl sarcosinates, alkyl phosphates, alkyl ether phosphates, alkyl ether carboxylates, alpha-olefin sulphonates, or combinations thereof.

17. A composition comprising:
(a) about 10 to about 40 wt. % of a hydrophobic polymer formed as a reaction product of a natural or food-derived oil and a methacrylate or acrylate polymer;
(b) about 1 to about 15 wt. % of one or more solvents capable of solubilizing the hydrophobic polymer of (a);
(c) about 20 to about 60 wt. % of a plurality of surfactants, wherein the plurality of surfactants comprises:
(c)(i) about 15 to about 30 wt. % one or more biosurfactants; and
(c)(ii) about 5 to about 20 wt. % one or more surfactants other than (c)(i);
(d) about 10 to about 50 wt. % of water;
wherein all percentages by weight are based on a total weight of the composition.

18. A cosmetic composition comprising:
(a) about 0.5 to about 10 wt. % of a hydrophobic polymer formed as a reaction product of a natural or food-derived oil and a methacrylate or acrylate polymer;
(b) about 0.1 to about 50 wt. % of one or more solvents capable of solubilizing the hydrophobic polymer of (a);
(c) about 0.5 to about 10 wt. % of a plurality of surfactants; and
(f) about 50 to about 95 wt. % of water;
wherein the composition is an oil-in-water emulsion, and all percentages by weight are based on a total weight of the composition.

19. The cosmetic composition of claim 17 comprising:
(a) about 0.5 to about 10 wt. % of a hydrophobic polymer formed as a reaction product of a natural or food-derived oil and a methacrylate or acrylate polymer;
(b) about 0.1 to about 20 wt. % of one or more solvents capable of solubilizing the reaction product of (a);
(c) about 1 to about 10 wt. % of a plurality of surfactants, wherein the plurality of surfactants comprises:
(c)(i) about 1 to about 8 wt. % of one or more biosurfactants;
(c)(ii) about 0.1 to about 6 wt. % of one or more surfactants other than (c)(i); and
(f) about 60 to about 90 wt. % of water;
wherein the composition is an oil-in-water emulsion, and all percentages by weight are based on a total weight of the composition.

20. A method for making a cosmetic composition comprising:
(i) forming an initial composition comprising:
(a) about 10 to about 40 wt. % of a reaction product of a natural or food-derived oil and a methacrylate or acrylate polymer;
(b) about 1 to about 30 wt. % one or more solvents capable of solubilizing the reaction product of (a);
(c) about 20 to about 60 wt. % of a plurality of surfactants; and
(d) about 10 to about 40 wt. % of water;
(ii) diluting the initial composition with water to form the cosmetic composition in the form of an oil-in-water emulsion.
